(19) United States
(12) Patent Application Publication
(10) Pub. No.: US 2024/0238214 A1
(43) Pub. Date: Jul. 18, 2024

(54) NANOCONSTRUCTS AND NANOPARTICLE-MEDIATED DELIVERY OF IMMUNOGENIC CELL DEATH INDUCERS FOR ENHANCING CANCER IMMUNOTHERAPY

(71) Applicant: Purdue Research Foundation, West Lafayette, IN (US)

(72) Inventors: Yoon Yeo, West Lafayette, IN (US); Soonbum Kwon, West Lafayette, IN (US)

(73) Assignee: Purdue Research Foundation, West Lafayette, IN (US)

(21) Appl. No.: 18/580,977

(22) PCT Filed: Jul. 20, 2022
(86) PCT No.: PCT/US2022/037727

§ 371 Date: Jan. 19, 2024

Related U.S. Application Data
(60) Provisional application No. 63/224,009, filed on Jul. 21, 2021.

(51) Int. Cl. A61K 9/51; A61K 31/337; A61K 38/07; A61K 39/395; A61P 35/00

(52) U.S. Cl. CPC A61K 9/5146; A61K 9/5123; A61K 9/5169; A61K 31/337; A61K 38/07; A61K 39/3955; A61P 35/00

(57) ABSTRACT

Nanoconstructs and compositions comprising a nanoparticle coated with an immunoadjuvant (e.g. ATP) and comprising one or more therapeutic agents (e.g. ICD inducer) encapsulated therein; and methods for treating cancer in a subject using such nanoconstructs and compositions, as well as combination immunotherapies.

### PRIORITY

The present application is related to and claims the priority benefit of U.S. Provisional Patent Application No. 63/224,009 filed Jul. 21, 2021, the content of which is hereby expressly incorporated by reference in its entirety into this disclosure.

### GOVERNMENT RIGHTS

This invention was made with government support under CA232419 and CA258737 awarded by the National Institutes of Health. The government has certain rights in the invention.

### TECHNICAL FIELD

This disclosure generally relates to nanoparticles, specifically poly(lactic-co-glycolic acid) nanoparticles loaded with an anti-cancer drug (e.g., an immunogenic cell death inducer) that is surface-modified with adenosine triphosphate, and methods for the treatment of cancer using such nanoparticles.

### BACKGROUND

Cancer is the second leading cause of death in the United States and takes the lives of more than 600,000 people each year. More than 1.9 million patients are expected to be diagnosed with cancer in the United States in 2022. While the lethality of cancer tumors has been trending down over the past several years, this is likely due to the development of the early detection techniques and improved lifestyles, rather than an increase in treatment efficacy. While various types of antitumor therapeutic treatments have been developed to treat cancer, limitations still exist due at least in part to patient variability and tumor heterogeneity.

Cancer immunotherapy has been explored in hopes of overcoming existing limitations by utilizing a patient's own immune system and has led to the development of various therapeutic agents. Generally, immunotherapy acts by selectively harnessing the host's immune defenses against the target, such as a cancerous tumor. Immune checkpoint blockade immunotherapy is one such type of immunotherapy that uses antibodies and other compounds to block T cell negative regulatory molecules (such as cytotoxic T-lymphocyte-associated protein 4 (CTLA-4) and anti-programmed cell death protein 1 (PD-1), for example) and has delivered positive results in some patients. However, due to the complex network of immunosuppressive pathways present in advanced tumors, only a minority of patients respond to this therapy.

Other cancer immunotherapies involve utilizing immunogenic cell death (ICD) inducers. ICD is a form of cancer cell death that can activate dendritic cells leading to tumor-specific T cell immunity. While it was long thought that cell death can be simply divided into only two different types of cell deaths, apoptosis and necrosis, recent findings suggest that more complexity exists. A third type of cell death, immunogenic apoptotic cell death, is different from the well-established concept of apoptosis (the silent removal of the cell) as immunogenic apoptotic cell death alarms the host's immune system and causes a cascade of immunological response. ICD inducers thus induce tumor cell death in such a way that exposes tumor antigens and is accompanied by endoplasmic reticulum stress and reactive oxygen species production in the cancer cells, which in turn induces the emission of signaling molecules known as damage-associated molecular patterns (DAMPs).

DAMPs serve as immunoadjuvants to activate antigen presenting cells (APCs), which are immune cells that specialize in presenting an antigen to a T-cell. The release of DAMPs can trigger the host immune system, which can confer a robust adjuvanticity to dying cancer cells. ICD-associated DAMPs can include surface-exposed calreticulin (CRT) as well as secreted adenosine triphosphate (ATP), annexin A1 (ANXA1), type I interferon, and chromatin-binding high mobility group B1 (HMGB1). Additional hallmarks of ICD encompass the phosphorylation of eukaryotic translation initiation factor 2 subunit-α (EIF2S1 or elF2α), the activation of autophagy, and a global arrest in transcription and translation.

Some chemotherapeutic agents (e.g., oxaliplatin, mitoxantrone, doxorubicin, bortezomib, and cyclophosphamide) can be ICD inducers. These agents carry the characteristics of ICD such that, when used to treat a tumor, they provide additional therapeutic effects due to sustained immune cell activation. However, to date, chemotherapeutic agents have had limited therapeutic efficacy due to the poor retention at the tumor. Moreover, ICD inducers exhibit high immunotoxicity, which remains a critical challenge when used alone or in combination with immune checkpoint inhibitors.

In addition, the tumor microenvironment (TME) can be regulated by multiple immunosuppressive cells, including tumor-associated macrophages (TAMs), myeloid-derived suppressor cells, cancer-associated fibroblasts, tumor-associated neutrophils, and regulatory T cells. For example, TAMs can comprise up to 50% of a solid tumor mass and interact with cancer cells and other immune cells to facilitate tumor growth through promoting angiogenesis, immunosuppression, and inflammation. The immunosuppressive TME can further complicate the efficacy of ICD-inducing chemotherapy.

In view of the above, the present disclosure seeks to provide a nanoparticle or compound that can encapsulate an ICD inducer (i.e., as the cargo), can increase the antigenicity of tumor cells, can recruit immune cells, and/or can be used in conjunction with other immunotherapies such as, for example, immune checkpoint blockade therapies.

### SUMMARY

Nanoconstructs are provided. In certain embodiments, a nanoconstruct comprises: a nanoparticle which has an exterior surface, one or more therapeutic agents encapsulated within the nanoparticle, and an immunoadjuvant modified polyphenol compound bound to the exterior surface of the nanoparticle.

The polyphenol compound can be selected from the group consisting of polymerized dopamine (pD), tannic acid, tannic acid-iron complex, gallic acid, ellagic acid, hydroxyhydroquinone, epigallocatechin, epicatechin gallate, epigallocatechin galate, and pyrogallol. In certain embodiments, the polyphenol compound is pD.

The immunoadjuvant can be selected from the group consisting of adenosine triphosphate (ATP), calreticulin, high motility group box 1, deoxyribonucleic acid, annexin A1, type I interferon, heat shock protein 70, and heat shock protein 90. In certain embodiments, the immunoadjuvant is ATP. In certain embodiments, the immunoadjuvant is calreticulin. In certain embodiments, the immunoadjuvant is high motility group box 1. The immunoadjuvant can be, for example, about 10.5 wt % to about 12.5 wt % of the nanoconstruct, or any other wt % of the construct as deemed desirable.

The nanoparticle can be selected from the group consisting of poly(lactic-co-glycolic acid) (PLGA), polycaprolactone, D-α-tocopherol polyethylene glycol 1000 succinate-PLGA conjugate, polylactic acid, PLGA-methoxy-polyethylene glycol, ethylene vinyl acetate, mesoporous silica, liposomes, nanocrystals, and polyphenol aggregates. In certain embodiments, the nanoparticle is a PLGA nanoparticle. In certain embodiments, the nanoparticle is a PLGA-methoxy-polyethylene glycol nanoparticle. In certain embodiments, the nanoparticle is a liposome. In certain embodiments, the nanoparticle is a nanocrystal.

The one or more therapeutic agents can be one or more chemotherapeutic agents. In certain embodiments, at least one of the one or more therapeutic agents is an immunogenic cell death (ICD) inducer. In certain embodiments, the one or more therapeutic agents can be is oxaliplatin, carfilzomib, paclitaxel, mitoxantrone, bleomycin, doxorubicin, epirubicin, idarubicin, cyclophosphamide, or cardiac glycosides (or any combination of the foregoing). In certain embodiments, the one or more therapeutic agents is carfilzomib. In certain embodiments, the one or more therapeutic agents is paclitaxel.

Compositions comprising the nanoconstruct are also provided. In certain embodiments, such a composition comprises a nanoconstruct and a pharmaceutically acceptable carrier.

Further, methods for treating a cancer in a subject are provided. In certain embodiments, a method for treating a cancer in a subject comprises administering to the subject a therapeutically effective amount of a nanoconstruct described herein or a composition described herein. The administering step can be, for example, performed subcutaneously, intravenously, intramuscularly, intraperitoneally, intratumorally, or topically.

The method can further comprise administering immunotherapy to the subject (e.g., a combination treatment method). In certain embodiments, the immunotherapy is immune checkpoint inhibitor therapy. The therapeutically effective amount of the nanoconstruct can be administered to the subject before administration of the immunotherapy to the subject. Alternatively, the therapeutically effective amount of the nanoconstruct can be administered to the subject after administration of the immunotherapy to the subject.

Methods are also provided for treating a cancer in a subject, such method comprising administering to the subject: a priming dose comprising a therapeutically effective amount of a nanoconstruct comprising a nanoparticle which has an exterior surface, one or more therapeutic agents encapsulated within the nanoparticle, and an immunoadjuvant modified polyphenol compound bound to the exterior surface of the nanoparticle; and an immune checkpoint inhibitor, a tumor-targeting antibody, or a cancer vaccine, wherein the priming dose induces or enhances an anti-tumor immune response in the subject at a targeted site. The targeted site can be a solid tumor. The targeted site is a cancerous tissue or cell.

The immunoadjuvant can be selected from the group consisting of adenosine triphosphate, calreticulin, high motility group box 1, deoxyribonucleic acid, annexin A1, type I interferon, heat shock protein 70, and heat shock protein 90. In certain embodiments, the one or more therapeutic agents are selected from the group consisting of oxaliplatin, carfilzomib, paclitaxel, mitoxantrone, bleomycin, doxorubicin, epirubicin, idarubicin, cyclophosphamide, and cardiac glycosides. In certain embodiments, the nanoparticle is selected from the group consisting of PLGA, polycaprolactone, D-α-tocopherol polyethylene glycol 1000 succinate-PLGA conjugate, polylactic acid, PLGA-methoxy-polyethylene glycol, ethylene vinyl acetate, mesoporous silica, liposomes, nanocrystals, and polyphenol aggregates.

The nanoparticle can be PLGA. The one or more therapeutic agents can be chemotherapeutic agents. At least one of the therapeutic agents can be an ICD inducer. The polyphenol compound of the method can be pD, tannic acid, tannic acid-iron complex, gallic acid, ellagic acid, hydroxyhydroquinone, epigallocatechin, epicatechin gallate, epigallocatechin galate, or pyrogallol. In certain embodiments, the polyphenol compound of the method can be selected from the group consisting of pD, tannic acid, tannic acid-iron complex, gallic acid, ellagic acid, hydroxyhydroquinone, epigallocatechin, epicatechin gallate, epigallocatechin galate, and pyrogallol. The immune checkpoint inhibitor can be an antibody or antibody fragment targeting PD-1 (e.g., nivolumab or pembrolizumab) or PD-L1 (e.g., atezolizumab, avelumab, or durvalumab), CTLA-4 (e.g., tremelimumab or ipilimumab), an anti-CD25 antibody (e.g., basiliximab), or decitabine (e.g., a demethylating agent to control T-cell exhaustion).

In certain embodiments, the priming dose of the method is administered at least four days prior to the immune checkpoint inhibitor, thereby treating cancer in the subject.

Methods for enhancing an anti-cancer immune response in a subject are also provided. In certain embodiments, such a method comprises administering to the subject a therapeutically effective amount of a nanoconstruct hereof or a composition hereof.

Still further, methods of enhancing the infiltration of immune cells into a targeted site are provided, such methods comprising: delivering one or more therapeutic agents to a targeted site in a subject, wherein delivery comprises administering a therapeutically effective amount of the nanoconstruct or the composition under conditions to deliver the nanoconstruct to the targeted site. In certain embodiments, the targeted site is a solid tumor and the nanoconstruct enhances an immune response (e.g., an antitumor-specific immune response) at the targeted site.

In certain embodiments, administering the therapeutically effective amount of the nanoconstruct or composition induces ICD at the targeted site. In certain instances, the targeted site is a solid tumor and administration of the therapeutically effective amount of the nanoconstruct or composition results in a decrease in volume of the solid tumor.

Administration of the therapeutically effective amount of the nanoconstruct or composition can result in remission of the tumor. In certain embodiments, administering is performed subcutaneously, intravenously, intramuscularly, intraperitoneally, intratumorally, or topically.

### DESCRIPTION OF THE FIGURES

The disclosed embodiments and other features, advantages, and aspects contained herein, and the matter of attaining them, will become apparent in light of the following detailed in description of various exemplary embodiments of the present disclosure. Such detailed description will be better understood when taken in conjunction with the accompanying drawings.

FIGS. 1A-1C show data related to the release of damage-associated molecular patterns (DAMPs) from CT26 cells treated with cytotoxic agents at IC₅₀. FIG. 1A shows data related to calreticulin (CRT) exposure on the CT26 cell surface. FIG. 1B shows data related to HMGB1 measured in the medium, and FIG. 1C shows data related to adenosine triphosphate (ATP) measured in the medium. *: p<0.05. ***: p<0.001. ns: no significant difference by one-way ANOVA with Dunnett's multiple comparisons test.

FIGS. 2A-2C show data related to the release of DAMPs from 4T1 cells treated with cytotoxic agents at IC₅₀, with FIG. 2A showing data related to CRT exposure on the 4T1 cell surface. FIG. 2B showing data related to HMGB1 measured in the medium, and FIG. 2C showing data related to ATP measured in the medium. *: p<0.05. ***: p<0.001, ns: no significant difference by one-way ANOVA with Dunnett's multiple comparisons test.

FIGS. 3A-3C show data related to the release of DAMPs from B16F10 cells treated with cytotoxic agents at IC₅₀, with FIG. 3A showing data related to CRT exposure on the B16F10 cell surface. FIG. 3B showing data related to HMGB1 measured in the medium, and FIG. 3C showing data related to ATP measured in the medium. *: p<0.05. ***: p<0.001, ns: no significant difference by one-way ANOVA with Dunnett's multiple comparisons test.

FIG. 4 shows the percent of CT26 cells phagocytosed by JAWSII DCs after treatment with CFZ or PTX for 6 hours or 24 hours (*: p<0.05. ***: p<0.001. ****: p<0.0001, ns: no significant difference by one-way ANOVA with Dunnett's multiple comparisons test).

FIG. 5 shows data from an in vivo vaccination study, with phosphate buffered saline (PBS) being the control, gemcitabine (GEM) being the negative control, oxaliplatin (OXA) being the positive control, and paclitaxel (PTX) and carfilzomib (CFZ) being the drug candidates.

FIG. 6 shows interferon gamma (IFN-γ) secretion data from splenocytes after stimulation with AHI peptide, a CT26 immunodominant MHC class-I restricted antigen.

FIG. 7A is a bar graph of the size and zeta potential of NP-pD-ATP measured in 10 mM phosphate buffer. pH 7.4. FIG. 7B is a plot of the change in ATP conjugation based on ATP to nanoparticle (NP) ratio (w/w). FIG. 7C is a transmission electron micrograph (TEM) of the NP-pD-ATP (visualized by negative staining with 1% uranyl acetate: scale bars: 100 nm).

FIG. 8A is a schematic illustration of the Transwell study setup with NP-pD-ATP or ATP.

FIGS. 8B and 8C are graphs of data collected from the study of FIG. 8A, with FIGS. 8B and 8C showing percent of THP-1 cells and JAWSII cells, respectively, that migrated across the Transwell (***: p<0.001, ns: no significant difference by one-way ANOVA with Dunnett's multiple comparisons test).

FIG. 8D shows the percent of JAWSII cells that migrated across the Transwell after various incubation times.

FIG. 8E shows the stability of PLGA-pD-ATP, estimated by the percentage of JAWSII cells that migrated across the Transwell in response to each treatment. Supernatant & pellet: Nanoparticles were pre-incubated in 10% FBS-containing medium for 24 hours and separated into supernatant and pellet (gray bars). The lack of JAWSII cell migration in response to supernatant indicates that ATP remained bound to nanoparticles and was not released to the supernatant.

FIG. 9A is a schematic illustration of the Transwell study setup with NP-pD-ATP or ATP with and without apyrase.

FIG. 9B is a graph of data collected from the study shown in FIG. 9A, with FIG. 10B showing percent of the JAWSII cells that migrated across the Transwell after treatment with apyrase (***: p<0.001, ns: no significant difference by one-way ANOVA with Dunnett's multiple comparisons test).

FIG. 9C is a bar graph of ATP levels measured using an ATP bioluminescence assay after co-incubation with apyrase.

FIG. 10 is a schematic illustration of ATP conjugation to PLGA nanoparticles. PLGA-NPs were coated with a polydopamine (pD) layer in pH 8.5 to produce PLGA-pD. The amine group of ATP was further conjugated to the hydroxyl group of pD.

FIGS. 11A and 11B are line graphs showing PTX and CFZ release kinetics, respectively, from the nanoparticles, each performed in PBS containing 0.2% Tween 80 with constant agitation at 37° C.

FIG. 12A shows a line graph of the CFZ release kinetics from the nanoparticles of the present disclosure, made of PLGA with an indicated lactic acid to glycolic acid (LA:GA) ratio and molecular weight (kDa). The study performed in PBS containing 0.2% Tween 80 with constant agitation at 37° C.

FIG. 12B shows the CFZ release kinetics from PLGA/PLGA-mPEG-NPs loaded with CFZ, the PLGA/PLGA-mPEG-NPs having different PLGA:PLGA-mPEG ratios (e.g., PLGA:PLGA-mPEG—100:0, 90:10, 50:50, and 0:100).

FIG. 12C shows the CFZ release kinetics from liposomes loaded with CFZ and produced at different pressures in a high-pressure homogenizer as described herein (with a pressure setting at 5000 psi and 20000 psi).

FIG. 12D shows liposome encapsulated CFZ release kinetics mass balance at various hours of the study.

FIGS. 13A and 13B show data plots from cytotoxicity studies assessing PTX (both free and in various encapsulated forms) in two different cell types (CT26 in FIG. 13A and JAWSII dendritic cell (DC) in FIG. 13B). PTX is free drug, PTX@NP is PTX encapsulated in an uncoated nanoparticle, PTX@NP-pD is PTX encapsulated in a pD-coated nanoparticle, and PTX@NP-pD-ATP is PTX encapsulated in a pD-coated nanoparticle decorated with ATP.

FIGS. 14A and 14B show data plots from cytotoxicity studies assessing CFZ (both free and in various encapsulated forms) in two different cell types (CT26 in FIG. 14A and JAWSII DC in FIG. 14B). CFZ is free drug, CFZ@NP is CFZ encapsulated in an uncoated nanoparticle, CFZ@NP-pD is CFZ encapsulated in a pD-coated nanoparticle, and CFZ@NP-pD-ATP is CFZ encapsulated in a pD-coated nanoparticle decorated with ATP.

FIG. 15A shows a graph of the changes in CT26 tumor size after the intratumoral injection of each treatment (equiv. to PTX 100 μg: n=3 per group), with measurements taken at various times over an 80-hour period post-injection.

FIG. 15B shows a bar graph representing percentages of CD11c⁺CD86⁺ DCs and the ratio of cells in a tumor after day 3 of each treatment, with *: p<0.05, **: p<0.01 by one-way ANOVA with Tukey's multiple comparison test. TME: Tumor microenvironment.

FIG. 16 is a data plot showing the changes in radiance of Cy7 from a tumor over time after a single intratumoral injection with each treatment (n=5 per group).

FIG. 17 shows images taken with an AMI whole body imager (Spectral Instruments, Inc., Tucson, Arizona) at various times following a single intratumoral injection of Cy7 dye to show changes in the radiance of the tumor over time.

FIG. 18 shows images taken with an AMI whole body imager at various times following a single intratumoral injection of NP-pD-Cy7 to show changes in the radiance of the tumor over time.

FIG. 19A is a schematic illustration of the procedure for how CT26 tumors were inoculated, and the treatments of the study were administered.

FIG. 19B shows a graph of changes in the CT26 tumor size following intravenous injection of each group (n=8 per group).

FIG. 19C shows a graph of the survival of the mice over time after the treatment.

FIG. 20A is a schematic illustration of the procedure for how the B16F10 tumors were inoculated and the treatments of the study were administered.

FIG. 20B shows a graph of the changes in tumor size after intravenous injection of each group (n=8 per group).

FIG. 20C shows a graph of the survival of the mice over time after the treatment.

FIG. 21A is a schematic illustration of the procedure for how the CT26 tumors were inoculated and the treatments of the study were administered (including the additional control groups of abxtal and abxtal+free ATP).

FIG. 21B shows a graph of changes in CT26 tumor size following intravenous injection of each treatment group (n=5 per group: CR=complete remission).

FIG. 21C shows a graph of the percent survival of the mice after the treatments (or lack thereof).

FIG. 22 is a schematic illustration of how the CT26 tumors were inoculated and the treatments of the study were administered (immune cells analyzed 7 days after intratumoral (IT) injection of each treatment).

FIG. 23 are data plots representative of the percent of immune cells and the ratio of cells in tumor at day 7 after the treatment (as described in connection with FIG. 22), wherein n=5: Mix: PTX@NP-pD+ATP: ATP-NP: PTX@NP-pD-ATP: *: p<0.05, **: p<0.01, ***: p<0.001, and ****: p<0.0001: and ns=no significant difference by one-way ANOVA with Tukey's multiple comparisons test.

FIG. 24 are data plots representative of the percent of immune cells and the ratio of cells in the tumor draining lymph nodes (TDLNs) at day 7 after the treatment (as described in connection with FIG. 22), where n=5, Mix=PTX@NP-pD+ATP: ATP-NP: PTX@NP-pD-ATP, and * is p<0.05, ** p<0.01: ** is p<0.01, *** is p<0.001, and **** is p<0.00001, ns=no significant difference by one-way ANOVA with Tukey's multiple comparison tests.

FIG. 25 are data plots representative of the percent of immune cells and the ratio of cells in the spleen at day 7 after the treatment (as described in connection with FIG. 22), n=5: Mix: PTX@NP-pD+ATP: ATP-NP: PTX@NP-pD-ATP: * is p<0.05, ** p<0.01: ** is p<0.01, *** is p<0.001, and **** is p<0.0001, ns=no significant difference by one-way ANOVA with Tukey's multiple comparison tests.

FIG. 26A are data plots representative of the percent of immune cells and the ratio of cells in the tumor at day 25 after the IT injection of each treatment and their correlation to the size of the tumor.

FIG. 26B are data plots representative of the percent of immune cells and the ratio of cells in the spleen at day 25 after IT injection of each treatment and their correlation to the size of the tumor.

FIG. 26C are data plots representative of the percent of immune cells and the ratio of cells in the spleen at day 25 after IT injection of each treatment and their correlation to the size of the tumor.

FIG. 27 is a schematic illustration of how the CT26 tumors were inoculated and the treatments of the study administered (anti-programmed cell death protein 1 (PD-1) antibodies given 7 days after the initial treatment). I.V.: Intravenous injection: I.P.: Intraperitoneal injection.

FIG. 28A is a graph of changes in CT26 tumor size after intravenous injection of PTX@NP-pD-ATP with (groups 2 and 4) or without (groups 1 and 3) intraperitoneal injection of anti-PD-1 antibodies (n=8 per group) pursuant to the protocol of FIG. 27.

FIG. 28B are individual growth curves of tumors after performing the treatment protocol of FIG. 27.

FIG. 29A is a graph of changes in CT26 tumor size after intraperitoneal injection of anti-PD-1 antibodies (n=8 per group) (with treatment starting when tumors were about 50-100 mm³ in volume).

FIG. 29B are individual growth curves of tumors after treatment with anti-PD-1 antibodies, with such treatment beginning when the tumors were small size tumors (about 50-100 mm³ in volume).

FIG. 30 shows a graph of percent tumor-free mice after rechallenge with CT26 tumor cells. The mice tested in this study included age-matched tumor naïve mice (tumor-naïve) and the mice that were treated with PTX@NP-pD-ATP and anti-PD-1 antibodies (NP+Abs) and reached complete remission in the combination therapy study of FIGS. 27-29B).

FIG. 31A shows a graph of the changes in CT26 tumor size after intravenous injection of PBS, PTX@NP-pD+ATP or PTX@NP-pD-ATP in nude mice (n=8 per group).

FIG. 31B shows a graph of the specific growth rate of tumors treated with PBS (control group), a mixed treatment (MIX: PTX@NP-pD+ATP), and an ATP-modified nanoparticle treatment (NP-ATP: PTX@NP-pD-ATP) (Δ log V/Δt).

FIG. 31C shows individual growth curves of the tumors in different treatment groups following treatment (**** is p<0.0001, ns=no significant difference by one-way ANOVA with Tukey's multiple comparison tests).

FIGS. 32A and 32B show confocal microscope images locating Rhodamine B-labeled nanoparticle coated with polyethyleneimine (PEI) or ATP in CT26 cells (FIG. 32A) and B16F10 cells (FIG. 32B), with green label: wheat germ agglutinin (cell membrane), red label: Rhodamine B-labeled nanoparticles, and blue label: DAPI (nuclei): scale bars: 50 μm.

FIG. 33 shows graphs of quantitative measurements demonstrating the amount of Rhodamine B labeled particles coated with PEI or ATP that are taken up by CT26 and B16F10 cells (measured via flow cytometry).

FIG. 34 shows a plot of the PTX content in CT26 tumors after 24 hours of a single intravenous injection of PTX@NP-pD+ATP or PTX@NP-pD-ATP (PTX equivalent 20 mg/kg) (n=5: ns: no significant difference by student t-test).

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the nanoparticles, compositions, and methods hereof can comprise many different configurations, forms, materials, and accessories.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

Nanoconstruct delivery platforms and compositions comprising such nanoconstructs are provided. In certain embodiments, the nanoconstructs are carriers of one or more therapeutic compounds (e.g., drugs or chemotherapeutic agents) and can enhance the retention and stability of such therapeutic compounds, target solid tumors for cancer treatment and/or enhance the infiltration of immune cells into a tumor (e.g., thus eliciting increased antitumor specific immune response) by surface modification with an immunoadjuvant. Accordingly, the nanoconstructs can be used to sensitize a tumor to anti-cancer therapies.

In certain embodiments, the nanoconstruct comprises a nanoparticle which has an exterior surface, one or more therapeutic agents encapsulated within the nanoparticle (e.g., cargo), and an immunoadjuvant modified polyphenol compound bound to the exterior surface of the nanoparticle.

In certain embodiments, the nanoconstruct comprises a poly(lactic-co-glycolic acid) (PLGA) nanoparticle that is surface-modified with an immunoadjuvant and encapsulates (e.g., is loaded with) one or more chemotherapeutic agents. An exemplary embodiment of such an immunoadjuvant is adenosine triphosphate (ATP).

In use, the immunoadjuvant (e.g., ATP) decorating the nanoparticle can recruit immune cells, such as antigen presenting cells (APCs), natural killer (NK) cells, and T cells, to a targeted site. Accordingly, administration of the nanoconstructs can result in enhanced infiltration of immune cells to a targeted site (e.g., a solid tumor), thereby activating (or escalating) the subject's specific immune response due to immune cell activation.

When used to treat cancer in a subject, such nanoconstructs can additionally deliver chemotherapeutic agents to the tumor site. The encapsulated therapeutic agent can be an immunogenic cell death (ICD) inducer. There, not only can the ICD inducer deliver anti-cancer drug therapy, but it can also increase the antigenicity of dying tumor cells at the target site, thus sensitizing them to other therapies (e.g., other immune-therapies). In certain embodiments, the nanoconstructs are administered in combination with an immune checkpoint inhibitor therapy to increase antitumor activity.

### Nanoconstructs

The nanoconstruct comprises a nanoparticle (NP) coated with one or more agents such as a polymer, a targeting molecule, a label, and/or a small molecule. The coated surface of the nanoparticle can further be decorated with one or more agents such as a targeting molecule, a label, an immunoadjuvant, and the like (e.g., ATP). One or more therapeutic agents can be encapsulated within the nanoparticle.

In use, nanoparticles can increase the retention of the cargo (e.g., drugs/therapeutic agents) in the targeted site (e.g., a tumor), exhibit enhanced pharmacokinetics, and be surface modified with a high surface to weight ratio to enhance the delivery of the cargo. See. e.g., Yu et al., Pharmacokinetics, biodistribution and in vivo efficacy of cisplatin loaded poly(L-glutamic acid)-g-methoxy poly(ethylene glycol) complex nanoparticles for tumor therapy, J Control Release 205: 89-97 (2015): Li et al., Efficient delivery of docetaxel for the treatment of brain tumors by cyclic RGD-tagged polymeric micelles, Molecular Med Reports 11: 3078-3086 (2015): Park et al., Polydopamine-based simple and versatile surface modification of polymeric nano drug carriers, ACS Nano 8: 3347-3356 (2014): Raymond et al., Oxaliplatin: mechanism of action and antineoplastic activity, Semin Oncol 25: 4-12 (1998).

The nanoparticle can comprise a polymer such as a biodegradeable polymer. In certain embodiments, the nanoparticle comprises one or more of poly(lactic-co-glycolic acid) (PLGA), polycaprolactone (PCL), PLGA-methoxy-polyethylene glycol (mPEG), D-α-tocopherol polyethylene glycol 1000 succinate (TPGS)-PLGA conjugate, polylactic acid (PLA), mesoporous silica, and ethylene vinyl acetate. In certain embodiments, the nanoparticle is a liposome and/or comprises a polyphenol aggregate. In certain embodiments, the nanoparticle is a drug crystal or nanocrystal, made mostly of drug molecules.

The nanoparticle can be a polymer selected to enhance the delivery of hydrophobic molecules by surface functionalization of the vehicle (i.e., nanoparticle). In certain embodiments, the nanoparticle is a PLGA nanoparticle.

Additionally, the nanoparticle can comprise two or more types of polymers, for example, a combination of PLGA and PLGA-mPEG at a w/w ratio of PLGA/PLGA-mPEG at about 90:10, about 85:15, about 50:50, about 35:65, about 65:35, about 15:85, or about 10:90. The ratio(s) of the polymer components can be adjusted to optimize the release kinetics of the resulting nanoparticle as is known in the relevant art.

The nanoparticles can be porous or nonporous. In certain embodiments, the nanoparticle is porous. In certain embodiments, the nanoparticle is substantially nonporous, but becomes porous over time (e.g., in use, when in circulation in vivo). The size of the nanoparticle cores can be from about 5 nm to about 200 nm, about 5 nm to about 20 nm, about 30 nm to about 100 nm, about 30 nm to about 80 nm, about 30 nm to about 60 nm, about 40 nm to about 80 nm, about 70 nm to about 90 nm, or about 5 nm, about 10 nm, about 20 nm, about 30 nm, about 40 nm, about 50 nm, about 60 nm, about 70 nm, about 80 nm, about 90 nm, or about 100 nm. In certain embodiments, the nanoparticle core size is suitable for systemic injection into a subject. Generally, the nanoparticle cores are spherical, although other shapes, such as rods and discs, can also be used.

Additional components can be attached to the nanoparticles by various mechanisms, covalently or noncovalently. In certain embodiments, the surface (or coated surface) of the nanoparticle is modified covalently through a chemical reaction (e.g., a Michael addition or Schiff base reaction). Alternatively, the surfaces of the nanoparticles can be altered to include reactive moieties for conjugation to ligands or other components as desired.

In certain embodiments, the nanoparticles, such as PLGA nanoparticles, are coated with one or more polymers or other compounds. This can be useful for functionalization purposes.

The polymer can bind to the surface of the nanoparticle using any appropriate means. In some embodiments, the polymer binds to the nanoparticle via a physical or chemical interactions. The polymer can be a polymer with a positive charge such as, without limitation, polyethylenimine (PEI). In some embodiments, the polymer is a polyphenol compound. In some embodiments, the polymer is polymerized dopamine (pD) or other polyphenols, such as tannic acid, tannic acid-iron complexes, gallic acid, ellagic acid, hydroxyhydroquinone, epigallocatechin, epicatechin gallate, epigallocatechin galate, and pyrogallol. In certain embodiments, the polymer is a cationic polymer (e.g., polyethylenimine).

In some embodiments, the nanoparticle comprises a pD coating. In some embodiments, the nanoparticle comprises a pD coating as a primer coating, onto which subsequent coating(s) is/are applied.

In certain embodiments, the nanoconstruct is a PLGA nanoparticle with a pD coating.

The polymer can be linear or branched. The polymer can be present from about 1 wt. % to about 50 wt. %, such as about 1 wt. % to 50 wt. % or 1 wt. % to about 50 wt. %, of polymer per nanoconstruct, e.g., 5 to 40%, 10 to 30%, 20 to 30%, 5 to 15%, 5 to 20%, 5 to 25%, 5 to 30%, 10 to 20%, 10 to 25%, or 25 to 40%, e.g., about 5, about 10, about 15, about 20, about 25, about 30, or about 35%.

The nanoconstruct can be surface modified (or functionalized) with a targeting moiety, an imaging agent, an immunoadjuvant, and/or another moieties to impart a particular characteristic to the nanoconstruct. Nanoparticles coated with pD in particular can accomodate ligands with nucleophilic functional groups via Michael addition and/or Schiff base reaction.

Examples of imaging agents include, without limitation, fluorescent dyes such as FITC, Cy dyes, and amine-reactive Alexa Fluor dyes. Other imaging agents that can be useful to decorate the surface of the nanoparticle will be apparent to one of ordinary skill.

The nanoconstruct can be configured for targeted delivery and/or controlled release of a therapeutic agent encapsulated therein. The nanoconstruct can comprise a targeting moiety, for example, for the targeted delivery of the nanoconstructs to particular cells or tissues (e.g., cell-interactive ligands to facilitate a cell-nanoparticle interaction). In certain exemplary embodiments, the nanoconstructs are configured for delivery to a specific site, such as a solid tumor, for therapy or treatment. The site can be in vivo, for example, in the case of a solid tumor present in a subject. Targeting agents can be used to target a site and optionally to aid or induce internalization into a cell.

The nanoparticle can be surface functionalized with an immunoadjuvant. In certain embodiments, the nanoparticle is modified with one or more chemokines useful to recruit dendritic or other activated immune cells including, without limitation, ATP, [N-[N-(4-methoxy-2,3,6-trimethylphenylsulfonyl)-L-aspartyl]-D-[4-amidino-phylalanyl)]-piperidine (CCR), C-X-C motif ligand (CXCL), and [(1R,4S,6S)-4-(4-amino-2-oxopyrimidin-1(2H)-yl)-6-hydroxycyclohex-2-en-1-yl]methyl dihydrogen phosphate (XCR). In certain embodiments, the immunoadjuvant can be ATP, calreticulin (CRT), high motility group box 1 (HMGB1), deoxyribonucleic acid (DNA), annexin A1 (ANXA1), type I interferon, heat shock protein 70 (HSP70), and heat shock protein 90 (HSP90).

In certain embodiments where immunoadjuvant comprises ATP, the ATP can be oriented in a manner that preserves the triphosphate group (e.g., for chemotactic activity) (see, e.g., Examples 4-6).

In certain embodiments, the nanoparticle is surface functionalized with CCR, which has the following structure:

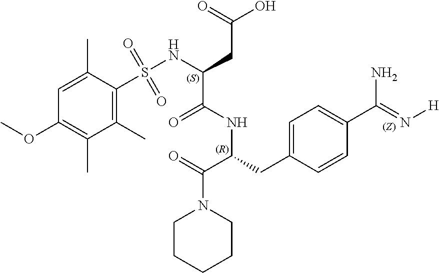

In certain embodiments, the nanoparticle is surface functionalized with XCR, which has the following structure:

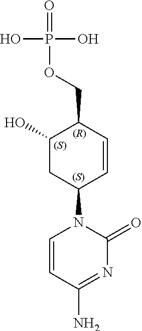

In certain embodiments, the nanoparticle is surface functionalized with ATP, which has the following structure:

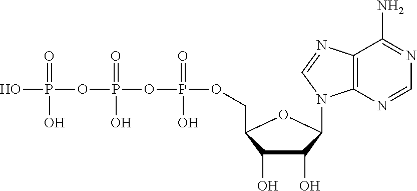

ATP is a chemoattractant of immune cells (e.g., dendritic cells and macrophages) and, among other functions, promotes the phagocytic clearance of dying cells. ATP contains an amine group, which can be conjugated to the surface of pD-coated nanoparticles.

The ATP-decorated nanoparticles provide stronger chemoattractive activity than free ATP (see Example 16) and maintain the chemotactic ability of ATP to recruit dendritic cells and macrophages to the targeted site (e.g., a tumor) (see Example 5), which translates into stronger activation of adaptive immune cells. Furthermore, the conjugation of ATP significantly improves the stability of ATP against apyrase (see Example 6), which prevents the degradation of ATP into the immunosuppressive products ADP, AMP and adenosine. Accordingly, conjugation of ATP to the nanoparticles not only maintains the activity of ATP but also improves its stability in vivo.

ATP can be present from about 1% to about 15% by weight (such as about 1% to 15% by weight, 1% to about 15% by weight, about 9% to about 11.5% by weight, about 9% to 11.5% by weight, or 9% to about 11.5% by weight). For example, ATP per PLGA can be used at the weight ratio ranging between about 10:1 to about 100:1 (such as about 10:1 to 100:1 or 10:1 to about 100:1, or about 9:1 to about 99:1, or about 15:1 to about 95:1, or about 15:1 to about 105:1) during the binding process, achieving complete binding.

In certain embodiments, the amount of conjugated ATP on the nanoparticle is about 10.5 wt % to about 11.2 wt % of the nanoconstruct (such as 10.5% to 11.2%). In certain embodiments, the amount of conjugated ATP on the nanoparticle is about 10.5 wt % to about 12.5 wt % (such as 10.5% to 12.5%) of the nanoconstruct. In certain embodiments, the PLGA per ATP can be used at the weight ratio of about 50:1, such as 50:1, during the binding process.

The nanoparticle can be loaded with one or more therapeutic agents. Such therapeutic agents can include, without limitation, chemotherapeutic agents such as ICD inducers (or compounds having the characteristics of ICD inducers), stabilizers, targeting agents, small molecules or proteins, labels, and/or oligonucleotides. Combinations of various additional agents are also contemplated. The nanoparticle can also include more than one type of polymer, stabilizer, targeting agent, small molecule, protein, label, oligonucleotide, mitoxantrone, bleomycin, doxorubicin, epirubicin, idarubicin, cyclophosphamide, and cardiac glycosides.

The nanoparticle can be loaded with one or more therapeutic agents comprising an ICD inducer. Nonlimiting examples of ICD inducers are carfilzomib, paclitaxel, oxaliplatin, mitoxantrone, bleomycin, doxorubicin, epirubicin, idarubicin, cyclophosphamide, and cardiac glycosides.

ICD inducers can be identified by detecting the release of immunostimulatory damage-associated molecular patterns (DAMPs), such as (i) surface exposure of CRT, (ii) passively released chromatin-binding HMGB1 and (iii) extracellularly secreted ATP. These molecules serve as “danger signals” for the immune system, making APCs recognize and process antigens to elicit adaptive immunity. CRT is a protein chaperone present in the endoplasmic reticulum. CRT is translocated to the membrane upon an insult, acting as an “eat me” signal to dendritic cells. HMGB1 is a non-histone chromatin-binding protein, which mediates DNA repair, recombination, and transcription. Extracellular HGMB1 binds to different types of pattern recognition receptors to promote cancer antigen processing and presentation by dendritic cells to T-cells. ATP is the main energy source in the cell, which is actively secreted during apoptosis. It works as a “find me” signal for APCs to promote phagocytic clearance of the dying cell.

The mechanism of action does not determine the ICD potential of a compound. For example, oxaliplatin is considered an ICD inducer, but cisplatin is not. Both are platinum compounds with the same mechanism of action (alkylating DNA to form intra-strand and inter-strand cross-links). Cisplatin differs from oxaliplatin by having an additional 1,2-diaminocyclohexane carrier ligand. A potential characteristic of an ICD inducer is the ability to induce endoplasmic reticulum stress, which can be a key indicator of an ICD inducer. Furthermore, ICD inducers can be, in certain instances, hydrophobic drugs that can benefit from encapsulation in a nanoparticle platform.

Carfilzomib (CFZ) and paclitaxel (PTX) are two such ICD inducers (see at least Examples 1-3). PTX is a microtubule inhibitor with low water solubility (8.5-17 μg/mL) and has the following structure:

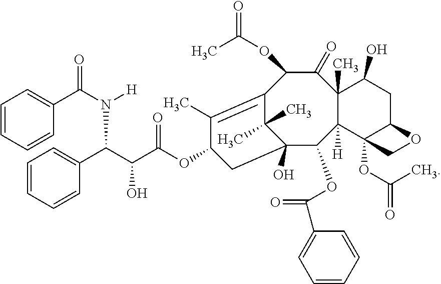

PTX causes cell cycle arrest in G2/M phase by interference with spindle formation, which induces cell death that can involve massive endoplasmic reticulum stress.

CFZ is a second-generation epoxyketone proteasome inhibitor with low water solubility (0.7-3.6 μg/mL) and has the following structure:

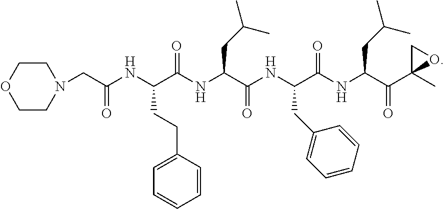 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

When administered, CFZ can establish irreversible and covalent interaction with the 20S proteasome of cancer cells, which can lead to the production of unfolded proteins causing endoplasmic reticulum stress.

ICD-inducing chemotherapy has recently gained interest as a way of enhancing cancer immunotherapy. ICD inducers increase the antigenicity of dying tumor cells, sensitizing them to immune checkpoint blockade therapies, for example. However, the immunotoxicity of ICD inducers (which can compromise the host's ability to mount antitumor immunity) and their insufficient adjuvanticity have conventionally remained critical challenges in combination therapy of ICD inducers and immune checkpoint inhibitors. The nanoconstructs can mitigate these issues by encapsulating the ICD inducers within the nanoparticle.

### Nanoconstruct Synthesis

The nanoconstructs can be prepared by conventional methods of organic synthesis practiced by those skilled in the art. The general reaction sequences outlined below in the Examples represent a general method useful for preparing the nanoconstructs and are not meant to be limiting in scope or utility.

Components can be bound to nanoparticles or other components by any means including covalent and non-covalent binding. Various conjugation chemistries are known in the art and described herein. In some embodiments, one or more of the components are bound to the surface of the nanoparticle (e.g., pD). In further embodiments, one or more of the components are bound to each other. For example, in some embodiments, a targeting moiety can be covalently bound to a stabilizer, which is covalently bound to a polymer (e.g., via an amine), which is in turn electrostatically bound to the exterior of the nanoparticle, and a therapeutic agent is bound to the interior surface of a pore.

While nanoparticles, such as PLGAs, can be acquired commercially or created by any method, in some embodiments, nanoparticles are formed by dissolving the polymer in the organic solvent, such as dichloromethane (DCM). The polymer solution is emulsified in an aqueous solution that contains emulsifiers, such as polyvinyl alcohol, which helps reduce the size of PLGA/DCM droplets. The particle size can be further reduced by the use of a probe sonicator or high-pressure homogenizer. In certain embodiments, the emulsion containing PLGA/DCM nanodroplets is subject to agitated evaporation (e.g., rotary evaporation) to remove DCM, resulting in solidified PLGA nanoparticles. The PLGA nanoparticles can be isolated by centrifugation and washed with DI water.

In some embodiments, the nanoparticles are coated with at least a polymer. The nanoparticles can be incubated with dopamine, for example, under oxidative conditions to yield a pD coated nanoparticle (nanoparticle-pD).

In some embodiments, functional groups and/or other components such as, without limitation, ATP can be added to the nanoparticles during synthesis by further incubating the coated nanoparticle with the components of interest (e.g., ATP) with one or more reagents as appropriate.

Therapeutic agents can also be encapsulated within the nanoparticles during or after synthesis. In certain embodiments, one or more therapeutic agents can be encapsulated in the coated and decorated nanoparticles by the single emulsion method with a target loading efficiency of 5 wt %, of 10 wt %, of 15 wt %, of 20 wt %, of 25 wt %, of 30 wt %, of 35 wt %, of 40 wt %, of 45 wt %, or 50 wt %, or any other wt % achievable and/or desired using the nanoconstructs hereof.

### Compositions, Routes of Administration and Dosing

Nanoconstructs can be formulated for therapeutic or research use. Typically, such formulations for therapy include the nanoconstructs suspended in a pharmaceutically acceptable carrier.

The nanoconstructs can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the host subject including, but not limited to, by intravenous, intratumoral, intramuscular, subcutaneous, transdermal, and like routes of administration.

As used herein, the term “composition” generally refers to any product comprising more than one ingredient, including the nanoconstructs.

The nanoconstructs can be formulated as pharmaceutical compositions and administered to a mammalian host, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. In at least one embodiment, nanoconstructs, such as part of a composition, can be administered directly into the blood stream, into muscle, into a solid tumor, or into an internal organ.

For example, in at least one embodiment, the nanoconstructs may be systemically administered in combination with a pharmaceutically acceptable vehicle. The percentages of the components of the compositions and preparations can vary and can be between about 1 to about 99% weight of the active ingredient(s) and a binder, excipients, a disintegrating agent, a lubricant, and/or a sweetening agent (as are known in the art). The amount of active compound (e.g., therapeutic agents) in such therapeutically useful compositions is such that an effective dosage level can be obtained.

The preparation of parenteral nanoconstructs/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the nanoconstructs/compositions can also be administered via infusion or injection (e.g., using needle (including microneedle) injectors and/or needle-free injectors). Solutions of the active composition can be aqueous, optionally mixed with a nontoxic surfactant and/or contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water or phosphate-buffered saline (PBS). For example, dispersions can be prepared in glycerol, liquid PEGs, triacetin, and mixtures thereof and in oils. Under ordinary conditions of storage and use, these preparations may further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants.

Sterile injectable solutions can be prepared by incorporating the nanoconstructs and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

For topical administration, it may be desirable to administer the nanoconstructs directly to a tumor site as compositions or formulations in combination with an acceptable carrier, which may be a solid or a liquid. For example, in certain embodiments, solid carriers may include finely divided solids such as talc, clay, microcrystalline cellulose, silica, alumina and the like. Similarly, useful liquid carriers may comprise water, alcohols or glycols or water-alcohol/glycol blends, in which the present compounds can be dissolved or dispersed at effective levels, optionally with the aid of non-toxic surfactants. Additionally or alternatively, adjuvants such as antimicrobial agents can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the terms “therapeutically effective.” “therapeutically effective dose,” or “therapeutically effective amount” (unless specifically stated otherwise) a quantity of a nanoconstruct and/or compound (e.g., a therapeutic agent) that, when administered either one time or over the course of a treatment cycle, affects the health, wellbeing or mortality of a subject (e.g., and without limitation, delays the onset of and/or reduces the severity of one or more of the symptoms associated with a cancer). In certain embodiments, a therapeutically effective amount can provide a prophylactic effect. Useful dosages of the nanoconstructs can be determined by comparing their in vitro activity with their in vivo activity in animal models. Methods of the extrapolation of effective dosages in mice and other animals to human subjects are known in the art. Indeed, the dosage of the nanoconstruct can vary significantly depending on the condition of the host subject, the cancer type being treated, how advanced the pathology is, the route of administration of the compound and tissue distribution, and the possibility of co-usage of other therapeutic treatments (such as radiation therapy or additional drugs in combination therapies). The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise. Therapeutically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg. 0.02 mg/kg. 0.03 mg/kg, 0.04 mg/kg. 0.05 mg/kg, 0.1 mg/kg. 0.2 mg/kg. 0.3 mg/kg. 0.4 mg/kg, 0.5 mg/kg. 1.0 mg/kg. 1.5 mg/kg. 2.0 mg/kg. 2.5 mg/kg. 3.0 mg/kg. 3.5 mg/kg. 4.0 mg/kg. 4.5 mg/kg, and 5.0 mg/kg, all of which are kg of patient body weight. The total therapeutically effective amount of the nanoconstruct can be administered in single or divided doses and can, at the practitioner's discretion, fall outside of the typical range given herein.

In certain embodiments, the nanoconstruct is loaded with PTX, formulated into a pharmaceutical composition for intravenous administration, and the therapeutically effective amount is about 15 mg/kg to about 30 mg/kg (such as 15 mg/kg to about 30 mg/kg or about 15 mg/kg to 30 mg/kg). In certain embodiments, the nanoconstruct is loaded with PTX, formulated into a pharmaceutical composition for intrathecal administration, and the therapeutically effective amount is about 2 mg/kg to about 10 mg/kg (such as 2 mg/kg to about 10 mg/kg or about 2 mg/kg to 10 mg/kg). In certain embodiments, the nanoconstruct is loaded with PTX and the therapeutically effective amount is 20 mg/kg for intravenous administration and 5 mg/kg for intrathecal administration.

In another embodiment, the nanoconstruct can be administered in a therapeutically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in single or divided doses and may, at the physician's discretion, fall outside of the typical range given herein. These amounts are based on meters of body surface area.

These and other effective unit dosage amounts can be administered in a single dose, or in the form of multiple daily, weekly or monthly doses, for example in a dosing regimen of twice per week for a 3-week cycle. In additional embodiments, dosages can be administered in concert with other treatment regimens in any appropriate dosage regimen depending on clinical and patient-specific factors. The amount, timing, sequence, and mode of delivery of compositions comprising a disease-treating effective amount (e.g., a therapeutically effective amount) of a nanoconstruct will be routinely adjusted on an individual basis, depending on such factors as weight, age, gender, and condition of the individual, the acuteness of the disease and/or related symptoms, whether the administration is prophylactic or therapeutic, and on the basis of other factors known to effect drug delivery, absorption, pharmacokinetics including half-life, and efficacy.

### Methods of Treatment and Use

Methods for delivering a therapeutic agent to a site in a subject using the nanoconstructs are provided. In certain embodiments, the method comprises administering a therapeutically effective amount of any of the embodiments of the nanoconstruct or compositions comprising same to a subject under conditions to deliver the nanoconstruct to a targeted site, wherein when the nanoconstruct is administered under the conditions, the nanoconstruct is internalized by the cell. The targeted site can be a solid tumor, a cancer, and/or a cancerous cell or tissue. In certain embodiments, the nanoconstruct elicits an enhanced antitumor-specific immune response in the targeted site. For example, the enhanced antitumor-specific immune response can be recruiting APCs, NK cells, and T cells to the targeted site.

In certain embodiments, the nanoconstruct enhances immune cell activation at the targeted site. For example, the ATP conjugated to the surface of the nanoconstructs can recruit tumor-specific cells to the targeted site and create an inflamed T cell-infiltrate tumor microenvironment (TME). In other words, the nanoconstructs, compositions and methods can aid in sensitizing the anti-cancer response and overcoming immune suppression by inducing the infiltration of NK cells, dendritic cells, and effector T cells into the TME.

In some embodiments, the accumulation of dendritic and other effector immune cells in the TME results in the uptake and presentation of tumor antigen by antigen-presenting cells (APCs) such as dendritic cells. The tumor antigen-laden dendritic cells subsequently migrate to tumor draining lymph nodes (TDLNs), where the dendritic cell continues to present the tumor antigen, thus inducing effector T cell activation, proliferation, and development.

In certain embodiments, administering the therapeutically effective amount of the nanoconstruct induces ICD in the targeted site or accelerates ICD in the targeted site (as compared to the rate of ICD without administration of the nanoconstruct).

Where the targeted site is a solid tumor, administration of the therapeutically effective amount of the nanoconstruct can result in a decrease in volume/size of the solid tumor. In certain embodiments, administration can result in remission of the tumor (e.g., complete remission of the cancer).

Methods for treating cancer in a subject are also provided. In certain embodiments, a method for treating cancer in a subject comprises administering to the subject a therapeutically effective amount of a nanoconstruct or composition comprising same. In certain embodiments, administration is performed subcutaneously, intravenously, intramuscularly, intraperitoneally, intratumorally, or topically.

The nanoconstruct and/or compositions comprising same can be delivered to a subject in conjunction with a means for sensitizing a tumor to anti-cancer therapy and/or other cancer treatments. In certain embodiments, the method further comprises administering to the subject immunotherapy (such as immune checkpoint inhibitor therapy). Such immunotherapy can be administered subsequently to or in conjunction with administration of a therapeutically effective amount of the nanoconstructs or compositions comprising same. The immunotherapy can be immune checkpoint inhibitor therapy.

Generally, immune checkpoint inhibitor therapy uses antibodies to block T cell negative regulatory molecules, such as CTLA-4 and PD-1. However, due to the complex network of immunosuppressive pathways present in advanced tumors and the TME, such therapies face challenges when used alone. Use of the nanoconstructs in conjunction with immune checkpoint inhibitor therapy contributes to an enhanced anti-tumor efficacy of the immune checkpoint inhibitor therapy administered after at least one nanoconstruct dose. Indeed, a single priming dose of the nanoconstructs can activate NK cells. T cells, and recruit APCs and dendritic cells to the targeted site. Moreover, these activated cells release cytokines to expand and further recruit additional immune cells to the targeted site, which results in the increased infiltration of cytotoxic T lymphocytes and the like in the TME and subsequently synergizes with the later administered immune checkpoint inhibitor therapy or other anti-cancer therapy.

Accordingly, in certain embodiments, a combination method for treating a cancer in a subject is provided. The method comprises administering to the subject a priming dose comprising a therapeutically effective amount of a nanoconstruct or composition comprising same: and a second therapy selected from the group consisting of an immune checkpoint inhibitor, a tumor-targeting antibody, and a cancer vaccine, wherein the priming dose induces or enhances an anti-tumor immune response in the subject at a targeted site. Where the second therapy comprises immune checkpoint inhibitor therapy, the immune checkpoint inhibitor can be an antibody or antibody fragment targeting PD-1 (e.g., nivolumab or pembrolizumab). PD-L1 (e.g., atezolizumab, avelumab, or durvalumab). CTLA-4 (e.g., tremelimumab or ipilimumab), an anti-CD25 antibody (e.g., basiliximab), or decitabine (e.g., a demethylating agent to control T-cell exhaustion). In certain embodiments, the priming dose of combination immunotherapy induces recruitment of NK cells and DC cells into the TDLNs.

Methods for enhancing an anti-cancer immune response in a subject are provided. The method can, in certain embodiments, comprise administering to the subject a therapeutically effective amount of a nanoconstruct or a composition.

Methods of enhancing the infiltration of immune cells into a targeted site are also provided. Such methods can comprise delivering one or more therapeutic agents to a targeted site in a subject, for example, by administering a therapeutically effective amount of the nanoconstructs or a composition comprising under conditions to deliver the nanoconstruct to the targeted site. The targeted site can be a solid tumor, for example, and the nanoconstruct can enhance an immune response (e.g., an antitumor-specific immune response) in the targeted site (e.g., by activating and recruiting immune cells to the site). In certain embodiments, administering the therapeutically effective amount of the nanoconstruct or composition induces ICD in the targeted site. Administration can be performed subcutaneously, intravenously, intramuscularly, intraperitoneally, intratumorally, topically or as otherwise desired and/or appropriate.

Administration of the therapeutically effective amount of the nanoconstruct or composition (e.g., in any of the methods) can result in a decrease volume of the cancer (e.g., solid tumor). In certain embodiments, administration of the therapeutically effective amount of the nanoconstruct or composition results in remission of the tumor.

### Certain Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl. Furthermore, unless specifically stated otherwise, the term “about” refers to a range of values plus or minus 10% for percentages and plus or minus 1.0 unit for unit values, for example, about 1.0 refers to a range of values from 0.9 to 1.1.

As used herein, the term “salts” and “pharmaceutically acceptable salts” refer to derivatives of the disclosed compounds wherein the parent compound is modified by making acid or base salts thereof. “Pharmaceutically” and “therapeutically” are used interchangeably herein. Examples of pharmaceutically acceptable salts include, but are not limited to, mineral or organic acid salts of basic groups such as amines: and alkali or organic salts of acidic groups such as carboxylic acids. Pharmaceutically acceptable salts include the conventional nontoxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two: generally, nonaqueous media like ether, ethyl acetate, ethanol, isopropanol, or acetonitrile are preferred. Lists of suitable salts are found in Remington's Pharmaceutical Sciences, 21st ed., Lippincott Williams & Wilkins, 2006, e.g., Chapter 38, the disclosure of which is hereby incorporated by reference.

The term “treating,” as used herein, encompasses therapeutic treatment (e.g., a subject with signs and symptoms of a disease state being treated) and/or prophylactic treatment. Prophylactic treatment encompasses prevention and inhibition or delay of progression of a disease state.

### EXAMPLES

The following examples serve to illustrate the present disclosure and are not intended to limit its scope in any way.

### Materials

The adenosine triphosphate (ATP) determination kit, the Cell Tracker Green and Cell Tracker Deep Red, Transwell® (8 μm pore size), ATP, dopamine HCL, and apyrase were all purchased from Thermo Fisher Scientific Inc. (Waltham, MA).

Poly(lactic-co-glycolic acid) (PLGA) (Lactel B6006-1, LA:GA=85:15, ester end, MW 97 kDA) was purchase from Sigma Aldrich (St. Louis, MO). All the other PLGA used were purchased from Akina Inc. (West Lafayette, IN).

(3-(4,5-Dimenthylthiazol-2-yl)-2, 5-diphenyltetrazolium bromide (MTT) was purchased from Thermo Fisher Scientific Inc. (Waltham, MA). Paclitaxel (PTX) was a gift of Samyang Biopharm Corporation (Seoul, Korea). Carfilzomib (CFZ) was purchased from Shenzhen Chemical Co. Ltd. (Shanghai, China). Oxaliplatin (OXA) was purchased from Thermo Fisher Scientific Inc. (Waltham, MA). Gemcitabine (GEM) was purchased from LC Laboratories (Woburn, MA).

CT26, B16F10, 4T1, JAWSII, THP-1 cells were purchased from the American Type Culture Collection (ATCC) (Manassas, VA). Dulbecco's modified Eagle medium (DMEM) and Roswell Park Memorial Institute (RPMI) were purchased from Thermo Fisher Scientific Inc. (Waltham, MA). Mouse granulocyte-macrophage colony-stimulating factor (GM-CSF) was purchased from Shenandoah Biotechnology, Inc. (Warminster, PA).

Fluorescein isothiocyanate (FITC) conjugated calreticulin (CRT) antibodies were purchased from Abcam plc (Cambridge, UK). High mobility group B1 (HMGB1) antibodies were purchased from Novus Biologicals (Littleton, CO). LEGEND MAX™ Mouse IFN-γ ELISA Kit was purchased from BioLegend (San Diego, CA).

Purified rat anti-mouse CD16/CD32 (Fc block) was purchased from BioLegend (San Diego, CA). Collagenase type 4, deoxyribonuclease I, and hyaluronidase were purchased from Worthington Biochemical Corporation (Lakewood, NJ). Anti-mouse CD16/32, CD45, CD11c, CD86, F4/80, CD206, NKp46, CD3, CD4, CD8, FOXp3, CD11b and GRI antibodies were purchased from BioLegend (San Diego, CA). Anti-mouse programmed cell death protein 1 (PD-1) antibody (CD279) was purchased from Bio X Cell (Lebanon, NH, USA). Female Balb/c mice (5-6 weeks old), male C57BL/6 mice (5-6 weeks old), and female athymic nude (Foxn1nu) mice (5-6 weeks old) were purchased from Envigo (Indianapolis, IN). Cy 7 amine was purchased from Lumiprobe (Cockeysville, MD).

All animal procedures were approved by Purdue Animal Care and Use Committee, in conformity with the National Institutes of Health (NIH) guidelines for the care and use of laboratory animals.

All the other materials were purchased from Thermo Fisher Scientific Inc. (Waltham, MA).

### Example 1

### In Vitro Evaluation of the Immunogenic Cell Death (ICD) Potential of PTX and CFZ

ICD inducers can be identified by detecting the release of immunostimulatory damage-associated molecular patterns (DAMPs), such as (a) surface exposure of CRT, (b) passively released chromatin-binding HMGB1, and (c) extracellularly secreted ATP. These molecules serve as “danger signals” for the immune system, making antigen presenting cells (APCs) recognize and process antigens to elicit adaptive immunity.

Therapeutic agents (carfilzomib (CFZ) and paclitaxel (PTX)) were tested against CT26 murine carcinoma, along with a positive control (oxaliplatin (OXA), a known ICD inducer) and a negative control (gemcitabine (GEM), a known non-ICD inducer). The release of DAMPs from the treated cells were then analyzed (e.g., CRT exposure was measured by flow cytometry, HMGB1 was measured by quantifying western blot, and ATP was measured by ATP determination kit).

CRT exposure: CT26 murine carcinoma, B16F10 murine carcinoma and 4T1 breast mammary carcinoma cells were each treated with either PBS (control), gemcitabine (GEM), oxaliplatin (OXA), paclitaxel (PTX), or carfilzomib (CFZ) at a concentration of IC₅₀ (half maximal inhibitory concentration of the relevant substance) for 6 hours. After 6 hours, cells were washed with PBS and fixed with 4% perfluoroalkoxy alkanes (PFA). Fixed cells were incubated with FITC-labeled CRT antibodies. Cells were analyzed by the Accuri C6 flow cytometer to determine the population of CRT-positive cells.

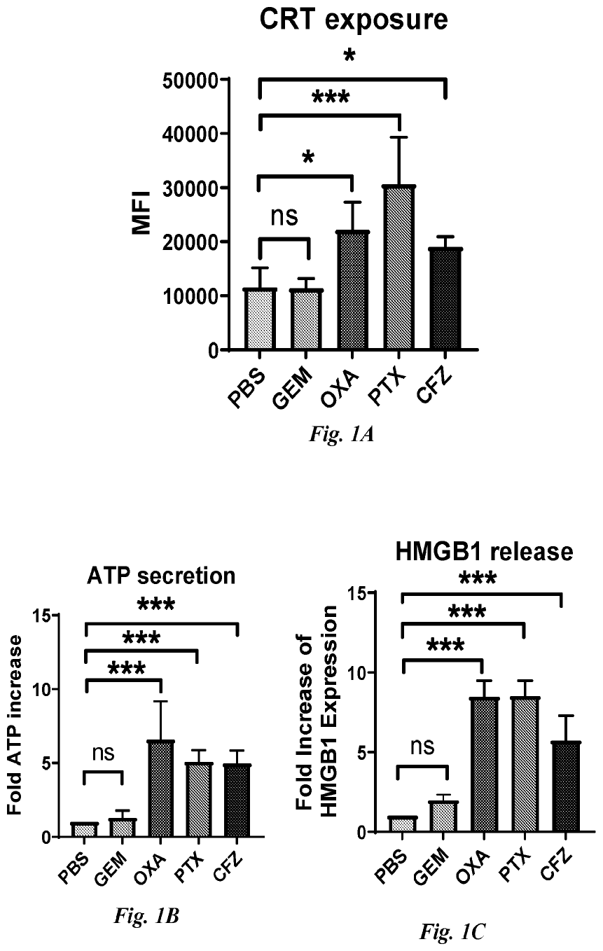

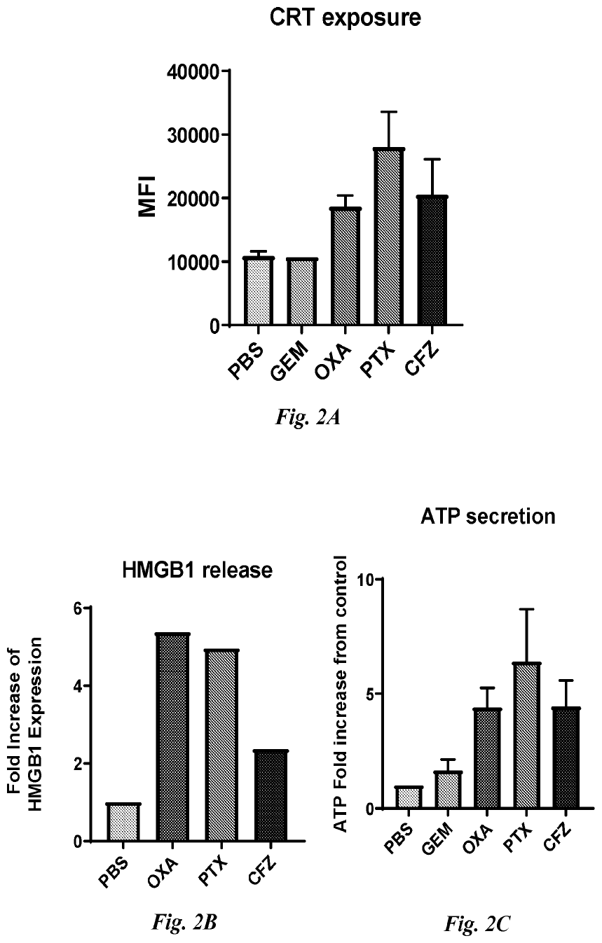

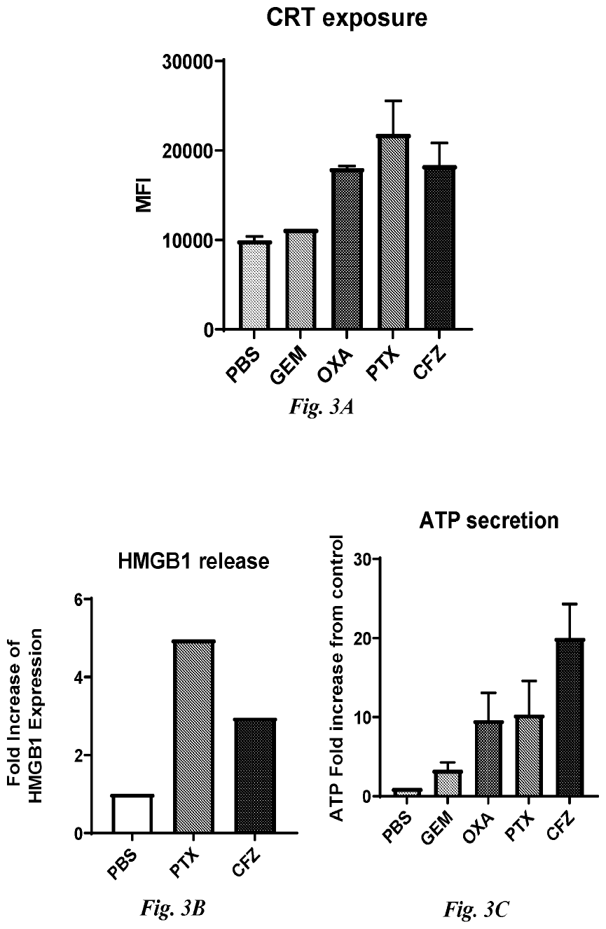

GEM failed to induce CRT exposure. When compared to the control groups, both PTX and CFZ induced the exposure of CRT (see FIGS. 1A, 2A, and 3A).

HMGB1 release: CT26 murine carcinoma, B16F10 murine carcinoma and 4T1 breast mammary carcinoma cells were each treated with GEM, OXA, PTX, or CFZ at a concentration of IC 50 for 24 hours. After 24 hours, supernatants were collected, and a western blot immunoassay was performed to determine the amount of HMGB1 present.

With respect to HMGB1, results similar to the CRT exposure were obtained, where OXA induced the release of HMGB1 and GEM failed to induce HMGB1 secretion. Both PTX and CFZ induced the release of HMGB1 (see FIGS. 1C, 2C, and 3C).

ATP secretion: CT26 murine carcinoma, B16F10 murine carcinoma and 4T1 breast mammary carcinoma cells were each treated with GEM, OXA, PTX, or CFZ at the concentration of IC₅₀ for 24 hours. After 24 hours, supernatants were collected, and the amount of ATP in the supernatant was measured with ATP determination kit. Both PTX and CFZ caused the secretion of ATP (see FIGS. 1B, 2B, and 3B).

Overall, the DAMPs screening supports that PTX and CFZ are ICD inducers, with both CFZ and PTX inducing comparable DAMP production. Various cell lines were used in this study to ensure that ICD induction occurs in the different cell lines.

### Example 2

### PTX and CFZ Enhance Phagocytosis

A phagocytosis assay was performed to observe whether cells killed by ICD inducers will be taken up by dendritic cells (DCs). As potential ICD inducers, PTX and CFZ were likely to enhance the uptake of dying tumor cells by dendritic cells.

CT26 cells were stained with Cell Tracker Green for 30 mins (0.5 μM) and washed once with cold serum free DMEM. CT26 cells were incubated overnight in 10% FBS media to allow any unbound dye to be inactivated. CT26 cells were collected and seeded (150,000 cells/well) in 6 wells plate for 24 hours. JAWSII cells were collected and stained with Cell Tracker Deep Red for 30 mins (0.5 μM) and washed once with cold serum free RPMI. JAWSII cells were incubated overnight in 10% FBS media to allow any unbound dye to be inactivated. After 24 hours of seeding, CT26 cells were treated with IC₅₀ of PTX (250 nM) or CFZ (15 nM) for 0 or 6 hours. After the treatment, CT26 containing 6 wells plate was centrifuged at 500 g×8 mins. Supernatant was aspirated to remove any remaining drugs. JAWSII cells (150,000 cells/well) were added to CT26, and cells were co-cultured for 24 hours. The co-cultured cells were collected, resuspended in the staining buffer, and analyzed by the Accuri C6 flow cytometer.

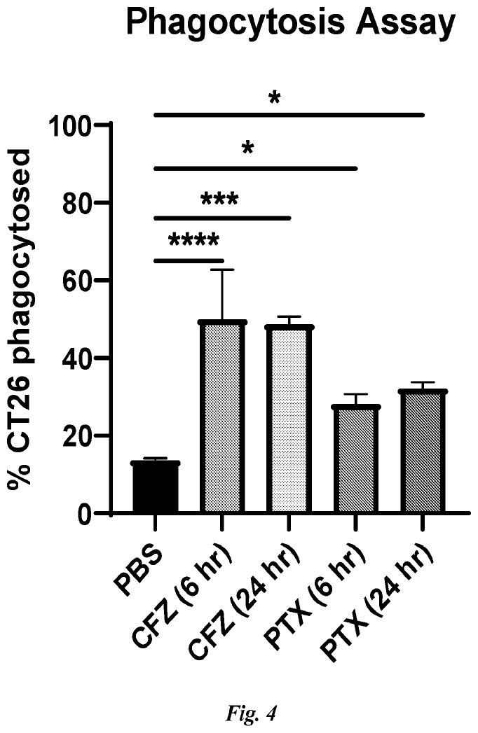

Treatment with either PTX or CFZ significantly increased the uptake of CT26 to the dendritic cells (see FIG. 4), which supports that treatment with PTX and CFZ can enhance phagocytosis and lead to strong antitumor immune response when used in vivo.

### Example 3

### In Vivo Tumor Vaccination Study

The vaccination study, a standard test to verify bona fide ICD, was performed with PTX and CFZ (e.g., the candidate drugs) in vivo. The vaccination study involved generating tumor antigens by exposing tumor cells to lethal dose of ICD inducers. Potential ICD inducer-treated cells (i.e., the primary dose) were injected subcutaneously to immunocompetent mice. One week later, live tumor cells were injected on the contralateral side (i.e., the rechallenge), and the growth of the tumor was monitored to determine if the drug-killed tumor cells (i.e., the primary dose) can serve as a vaccine and prevent the growth of a live cell challenge.

Here, CT26 cells were cultured to 30% confluency in a T150 flask and treated with OXA, GEM, PTX or CFZ at 100× IC₅₀ for 24 hours to create an ex vivo vaccine. Dead cells were collected and washed with PBS twice to remove excess amount of the drug.

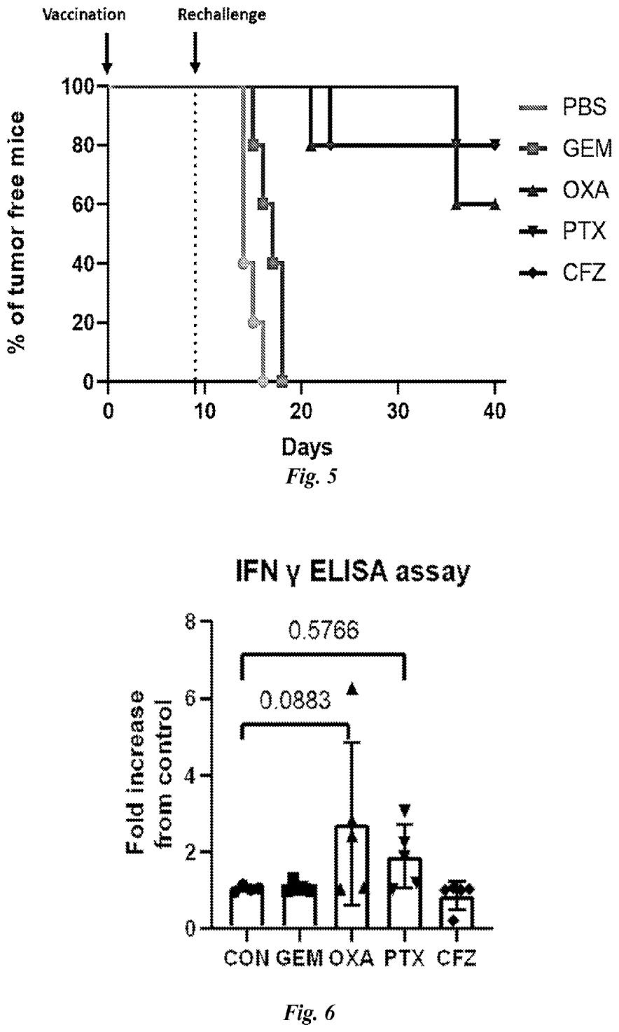

Three million dead cells were injected into the right flanks of 6-week-old immunocompetent female Balb/c mice (i.e., the primary dose), the syngeneic host of CT26 cells. After 9 days, 500,000 live CT26 cells (live tumor cells) were inoculated subcutaneously in the contralateral side of the mice (i.e., the rechallenge) and tumor growth was monitored to determine the development of anti-tumor immunity. Tumors grew in all animals that received PBS or GEM-treated cells (FIG. 5). In contrast, tumors did not grow in animals vaccinated with OXA, PTX and CFZ-treated cells, supporting that the drug-treated tumor cells (i.e., the primary dose) served as a tumor vaccine and established anti-tumor immunity.

To test if the anti-tumor immunity was tumor-specific, splenocytes obtained from the vaccinated mice at the conclusion of the study of Example 2 were challenged with AHI peptide, a CT26 immunodominant MHC class-I restricted antigen, and evaluated with respect to interferon gamma (IFN-γ) production (FIG. 6).

Briefly, spleen was collected, minced and passed through a 100 μm filter, followed by a 40 μm filter. Single cell suspension was treated with ammonium-chloride-potassium (ACK) lysis buffer, then washed. Splenocytes were seeded into a 96 well plate (300,000 cells per well) and stimulated with 10 μg/mL of AHI peptide in presence of 20 ng/ml of GM-CSF. Cells were incubated for 72 hours and then centrifuged down to collect the supernatant. The concentration of IFN-γ in the collected supernatants was measured using an ELISA kit.

The vaccination study supports that PTX and CFZ are ICD inducers.

### Example 4

### ATP Coated PLGA Nanoparticle (NP-pD-ATP) Characterization

PLGA was chosen for the vehicle of ICD inducers, which is a biodegradable polymer that can be utilized to enhance the delivery of hydrophobic molecules by surface functionalization of the vehicle. A PLGA nanoparticle can be coated with polydopamine (PD), for example, and functionalized to increase the recruitment of DCs. ATP was selected as a potential candidate for a chemokine that recruits DCs because it contains an amine group, which can be conjugated to the surface of pD-coated nanoparticles.

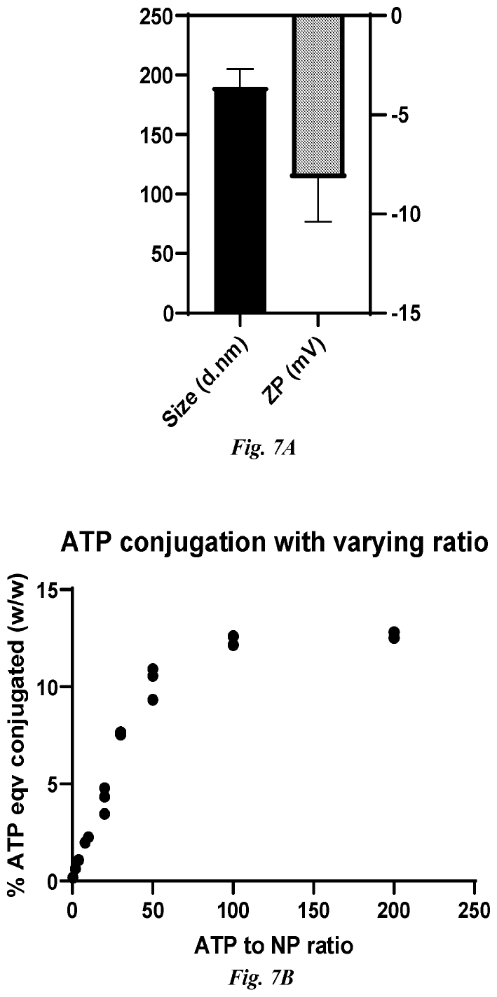

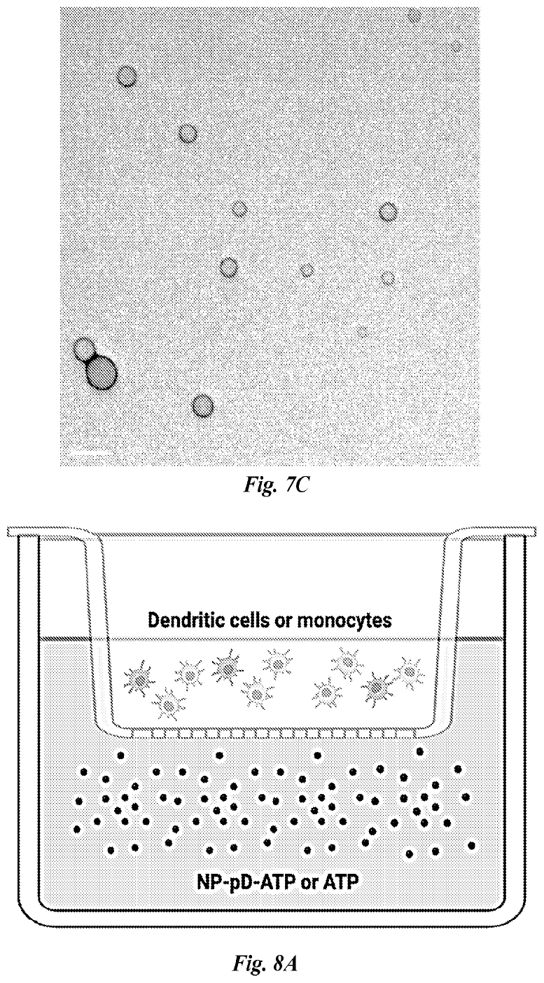

PLGA nanoparticles were incubated with dopamine under the oxidative condition to yield pD coated PLGA nanoparticle (nanoparticle-pD). NP-pD was further incubated with ATP to produce ATP coated nanoparticles (NP-pD-ATP) (see FIG. 7A and FIG. 7C). The amount of conjugated ATP on the NP-pD-ATP was measured with an ATP bioluminescence assay, and the ATP conjugation was 10.5 to 11.2 wt %. The maximum amount of conjugation (12.5% w/w) was achieved at the ATP to nanoparticle w/w ratio of 50:1 (FIG. 7B).

### Example 5

### Immunoactivity of NP-pD-ATP

It is important for ATP to maintain its chemoattractant activity after conjugation to the nanoparticle. The chemotactic ability of ATP is linked to the triphosphate group: thus, conjugating ATP via the amine group is expected to not affect the chemotactic function of ATP. The chemotactic ability of ATP or NP-pD-ATP (i.e., the ability of ATP or ATP-decorated nanoparticles to recruit DCs) was assessed using THP-1 human monocytes or JAWSII murine DCs in a Transwell. The two cell lines represent cell populations responsible for initiating immune responses: monocytes serve as a precursor to DCs, which present antigens to T-cells.

In the top well of the Transwell (8 μm pore size), monocytes (100 μL of 2×10⁵ THP-1 cells) or DCs (100 μL of 2×10⁵ JAWSII cells (immune cells of an immortalized immature DC line)) suspended in media were added. 600 μL of media containing 10 μM ATP or ATP-conjugated-nanoparticle (NP-pD-ATP) was added to the bottom wells. Cells were be incubated in 37° C. for 4 hours. After 4 hours of incubation, bottom media was collected and the number of monocytes or DCs that migrated from top well to the bottom well was counted using Accuri C6 flow cytometry.

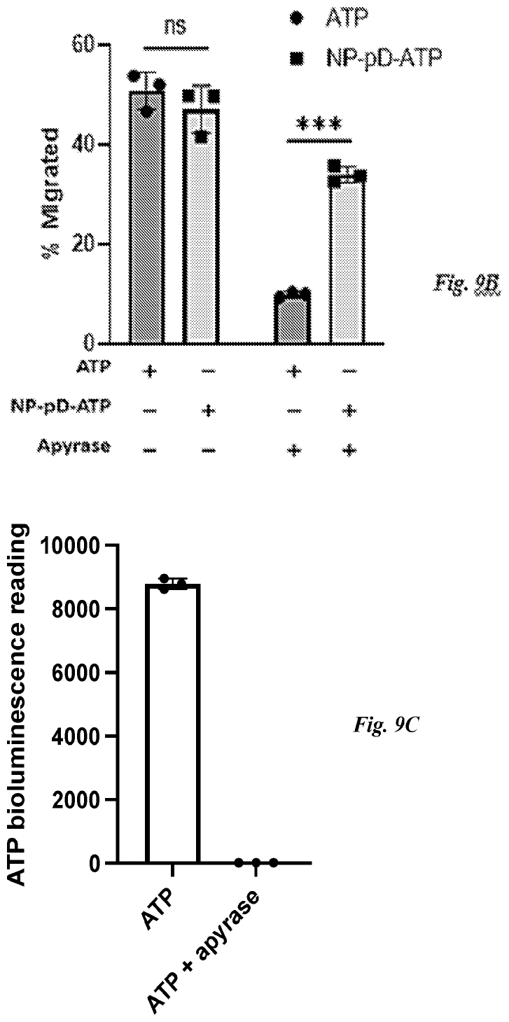

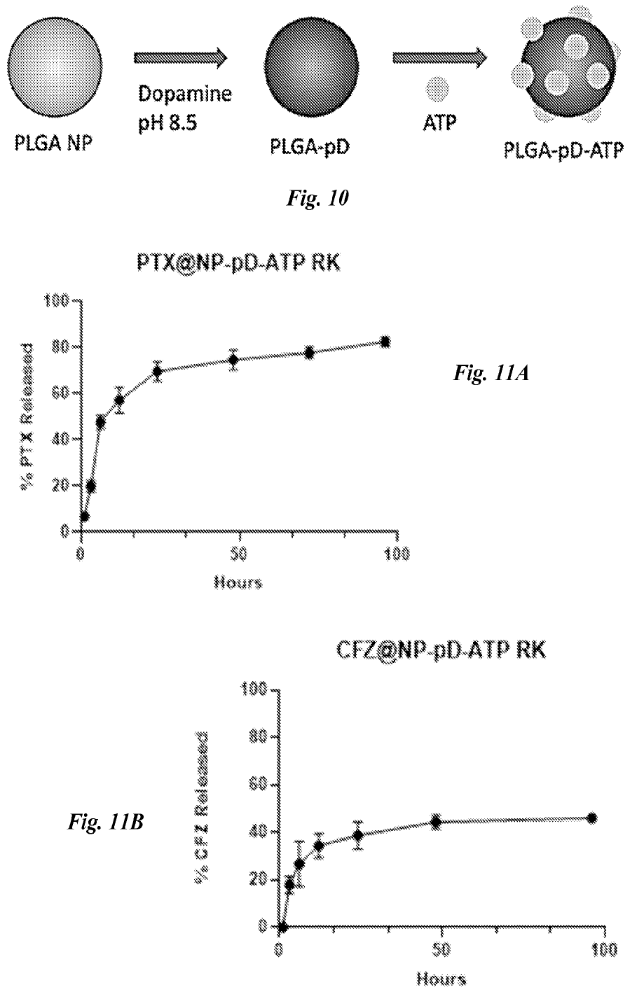

Based on the Transwell assay, NP-pD-ATP maintained the activity of ATP, and the chemoattractant activity was comparable to that of free ATP (see FIG. 9A). Based on the Transwell assay, NP-pD-ATP maintained the activity of ATP and the chemoattractant activity was comparable to that of free ATP (see FIGS. 9B and 9C).

### Example 6

### Stability Assessment of NP-pD-ATP

The stability of the NP-bound ATP was assessed by incubating the NP-pD-ATP (i) in a serum containing media, or (ii) in the presence of apyrase, an ATP-degrading enzyme.

### NP-pD-ATP is Stable in Serum-Containing Media

NP-pD-ATP equivalent to 1 mg ATP was incubated in 1 mL 10% fetal bovine serum (FBS) and 90% DMEM for up to 72 hours. At different time points during the incubation (e.g., 6 hours, 12 hours, 24 hours, 48 hours, and 72 hours), the nanoparticles were separated from the supernatant by centrifugation 25000 rcf×15 minutes, and the nanoparticles and supernatants were collected. The incubated nanoparticles and serum containing medium (that used to contain NP-pD-ATP) were used for a Transwell assay with JAWSII DCs.

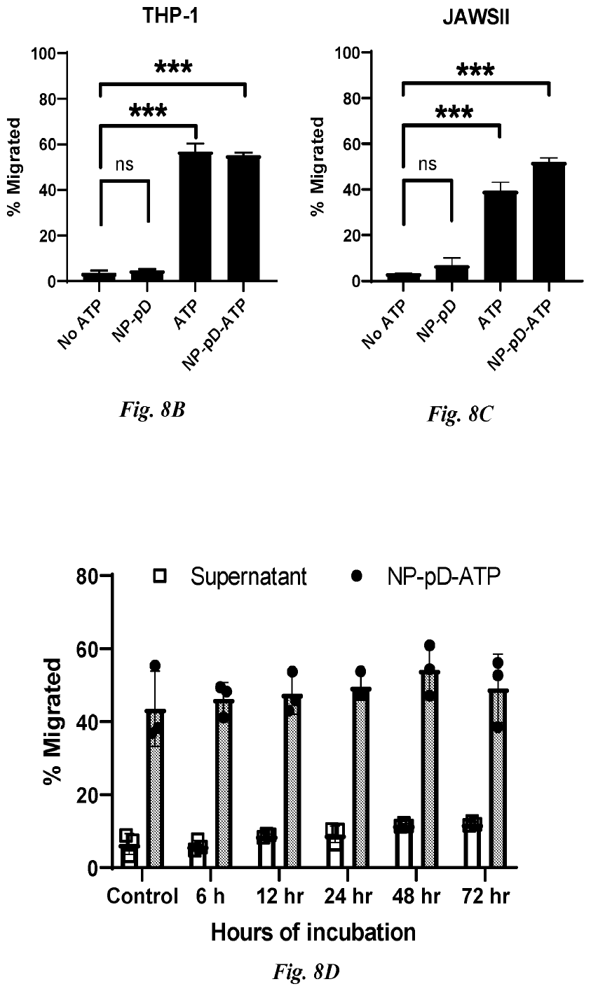

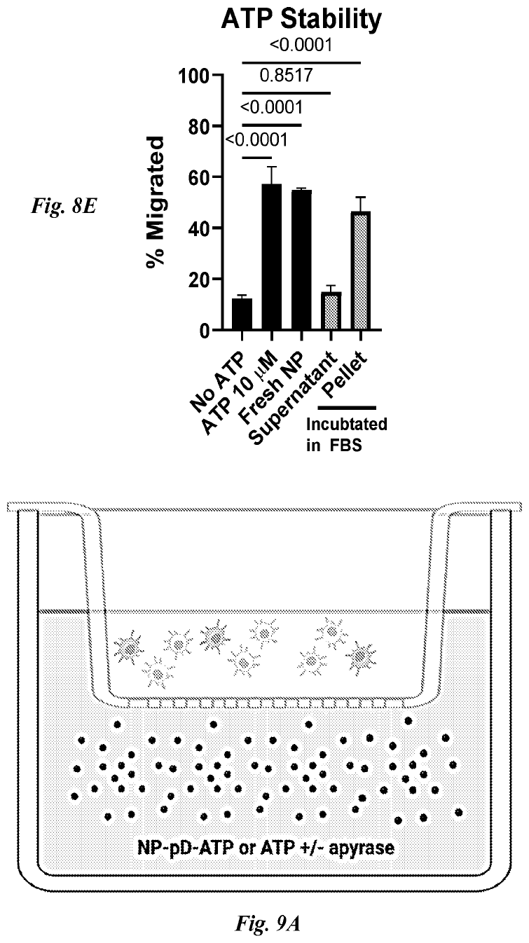

Briefly, nanoparticles were resuspended in 1 mL PBS. 10 μM ATP equivalent amount of NP-pD-ATP of the acquired supernatant from the incubated media was added to the bottom well of the Transwell. NP-pD-ATP, after 72 hours of incubation, still maintained the activity of ATP, while the particle conditioned supernatant did not induce the migration of the JAWSII DCs. This supports that ATP was stably bound to the nanoparticles, even in the presence of the serum (FIG. 8D).

FIG. 8E shows the stability of PLGA-pD-ATP estimated by the percentage of JAWSII cells that migrated across the Transwell in response to each treatment, including nanoparticles pre-incubated in 10% FBS-containing medium for 24 hours. Specifically, nanoparticles were pre-incubated in 10% FBS-containing medium for 24 hours and separated into supernatant and pellet. The lack of JAWSII cell migration in response to supernatant indicates that ATP was not released to the supernatant. Moreover, the nanoparticle pellet induced JAWSII migration to a comparable extent as fresh nanoparticles, indicating that the majority of ATP remained bound to the nanoparticles.

### NP-pD-ATP is Stable in Apyrase

While ATP may serve as a chemokine stimulating the immune system, its degraded products (i.e., adenosine diphosphate (ADP), adenosine monophosphate (AMP) and adenosine) each serve an opposite role, inducing an immunosuppressive environment. This mechanism is utilized in our body to prevent the unnecessary chronic inflammation. Accordingly, if bound ATP hydrolyzes into ADP, AMP or adenosine, the DC recruitment would be hindered and create an immunosuppressive environment promoting the tumor growth. ATP degrading enzymes such as CD39 and CD73 are commonly expressed on the surface of tumor: therefore, understanding how ATP behaves in presence of the enzyme is critical.

To screen the effect of degrading enzyme to the nanoparticle-bound ATP, apyrase (a CD39 equivalent) was co-incubated with ATP or NP-pD-ATP, and a DC migration assay was performed in a Transwell (FIG. 9A). For the migration assay, 100 μL of 2×10⁵ JAWSII cells, suspended in media, were added to the top of the Transwell (8 μm pore size). 600 μL of media containing 10 μM ATP conjugated nanoparticle was added to the bottom well (with or without 0.5 units of apyrase). Cells were incubated in 37° C. for 4 hours. Bottom media was collected to analyze the number of cells migrated by flow cytometry.

Further, to confirm the activity of apyrase against ATP, 10 μM ATP was incubated with or without 5 units/mL apyrase in 37° C. for 10 minutes. The ATP level after incubation was measured by ATP bioluminescence assay.

In a Transwell migration assay, apyrase completely abolished the chemotactic ability of free ATP, as shown by significantly reduced migration of DC (FIG. 9B). However, NP-PD-ATP resisted the degradation of apyrase and induced the migration of DCs to the bottom well. The surface bound ATP may have induced a steric hinderance to the apyrase enzymes, preventing the degradation of the ATP.

In addition, ATP was not detectable after free ATP incubation with apyrase, supporting the activity of apyrase to catalyze the sequential hydrolysis of ATP to ADP, AMP, and eventually adenosine to release inorganic phosphate. (FIG. 9C).

Overall, the results of stability experiments suggest that ATP was stably conjugated to the surface of nanoparticles, and such conjugation increased the stability of ATP against its degrading enzyme.

### Example 7

### Encapsulation of ICD Inducers in Nanoparticles

After the development of NP-pD-ATP, ICD inducers were encapsulated. PTX or CFZ was encapsulated in PLGA nanoparticles by the single emulsion method with a target loading efficiency of 5 wt %. PLGA nanoparticles were chosen for the study due to their compatibility with hydrophobic drugs and their high surface area to weight ratio that facilitates the conjugation of functional ligands.

100 mg of PLGA (MW 97 kDa, LA:GA=85:15) and 10 mg of PTX or CFZ was dissolved in 1 mL of dichloromethane. The solution was added to 4 mL of 4% polyvinyl alcohol (PVA). The mixture was emulsified by sonication for 2 minutes (4 seconds on, 2 seconds off, 40% amplitude). 20 mL of DI water was added to the emulsion. The solution was stirred for 3 hours, then further evaporated with rotavapor for additional 30 minutes.

Nanoparticles loaded with PTX cargo or nanoparticles loaded with CFZ cargo were collected via centrifugation (45 k rcf×20 minutes) and coated with a pD layer to allow for the subsequent conjugation of ATP to the pD via Michael addition and Schiff base reaction (FIG. 11). Collected particles were washed with deionized (DI) water twice. PTX loaded PLGA nanoparticles (PTX@NP) were incubated in dopamine HCl solution in Tris buffer (10 mM, pH 8.5) for 2 hours at a dopamine HCl-to-NP weight ratio of 0.5/1. When nanoparticles manifested dark color of polymerized dopamine, nanoparticles were collected by centrifugation (20 minutes×25000 rpm) and washed twice with DI water to remove excess dopamine and pD.

PTX loaded NP-pD (PTX@NP-pD) were incubated with ATP at an ATP to nanoparticle weight ratio of 20:1 for 1 hour in Tris buffer (10 mM, pH 8.5). The nanoparticles were collected by centrifugation at 45 k ref for 20 minutes at 4° C. and washed twice with DI water. The size was measured by NS90 zetasizer, zeta potential was measured by NS90 zetasizer, the loading efficiency was measured by high-performance liquid chromatography (HPLC), and the ATP content was measured using an ATP determination kit. Dipalmitoylphosphatidylcholine (DPPC) and cholesterol were purchased from Avanti Polar Lipids, Inc. (Birmingham, AL).

The size and surface charge of drug-encapsulated nanoparticles were measured by the Malvern NS90 zetasizer. The loading efficiency was defined as drug (quantified by HPLC) per nanoparticle. PTX and CFZ were encapsulated in PLGA nanoparticles with loading efficiency of 3.4 and 4.4%, respectively. Drug-encapsulated nanoparticles had particle sizes suitable for systemic injection (Table 1).

TABLE 1
Drug-loaded nanoparticle characterization

| | | Zeta | Loading | |
|---|---|---|---|---|
| | Z- Average | potential | Efficiency | ATP content |
| Compound | (d · nm) | (mV) | (w/w %) | (w/w %) |
| PTX@NP-pD-ATP | 190 ± 15 | −8.2 ± 2.2 | 3.4 ± 1.0 | 10.5 |
| CFZ@NP-pD-ATP | 191 ± 20 | −7.5 ± 1.0 | 4.4 ± 1.1 | 11.2 |

### Example 8

Release Kinetics from NP-pD-ATP

The release kinetics of PLGA-pD-ATP loaded with PTX and PLGA-pD-ATP loaded with CFZ were measured in 0.2% Tween 80/PBS under the sink conditions. The saturation solubility of PTX and CFZ in 0.2% Tween 80/PBS was determined to be 13 μg/mL and 9 μg/mL, respectively. The supernatant was collected, and the amount of the drug was measured with HPLC at each time indicated in FIGS. 11A and 11B.

As shown in FIGS. 11A and 11B, PTX@NP-pD-ATP released 82.5% of total loaded drug over 96 hours, and CFZ@PLGA-pD-ATP released 46%. Initial burst release was observed for both drugs to different extents, likely due to leakage of the drug located near the particle's surface and a poorly entrapped drug that easily diffuses out. The delayed and incomplete release of CFZ from CFZ@PLGA-pD-ATP is possibly due to the poor solubility of the compound in FBS containing medium, which was previously observed.

### Example 9

### Enhancing Release of the Encapsulated Therapeutic Agent

Various studies were performed to assess the effect of variables on the release kinetics of the ATP-decorated nanoparticles. Primarily, because CFZ encapsulated in PLGA did not provide a release of more than 50% of the loaded cargo, further investigations were performed to provide a complete release.

### Alternation of Lactic Acid to Glycolic Acid (LA:GA) Ratio or Molecular Weight (MW)

First, the LA:GA ratio or MW of the PLGA polymer was varied to provide either a hydrophobic or hydrophilic environment for CFZ. Increasing the hydrophobicity has the potential to improve the release kinetic, as CFZ is hydrophobic and may be molecularly dispersed in PLGA matrix. As a consequence of molecular dispersion, CFZ may not be crystalized, thereby increasing the release. On the other hand, decreasing the hydrophobicity by altering LA:GA ratio can help release CFZ by increasing water infiltration. Nanoparticles were produced with varying LA:GA ratio and MW (Table 2).

TABLE 2
Alteration of LA:GA ratio and MW

| | LA:GA | MW | Changes made compared to |
|---|---|---|---|
| Sample | ratio | (kDA) | the standard |
| Standard | 85:15 | 97 | — |
| 1 | 85:15 | 45 | ↓MW |
| 2 | 85:15 | 150 | ↑MW |
| 3 | 60:40 | 100 | ↑LA ratio |
| 4 | 60:40 | 37 | ↑LA ratio, ↓↓MW |
| 5 | 60:40 | 65 | ↑LA ratio, ↓MW |

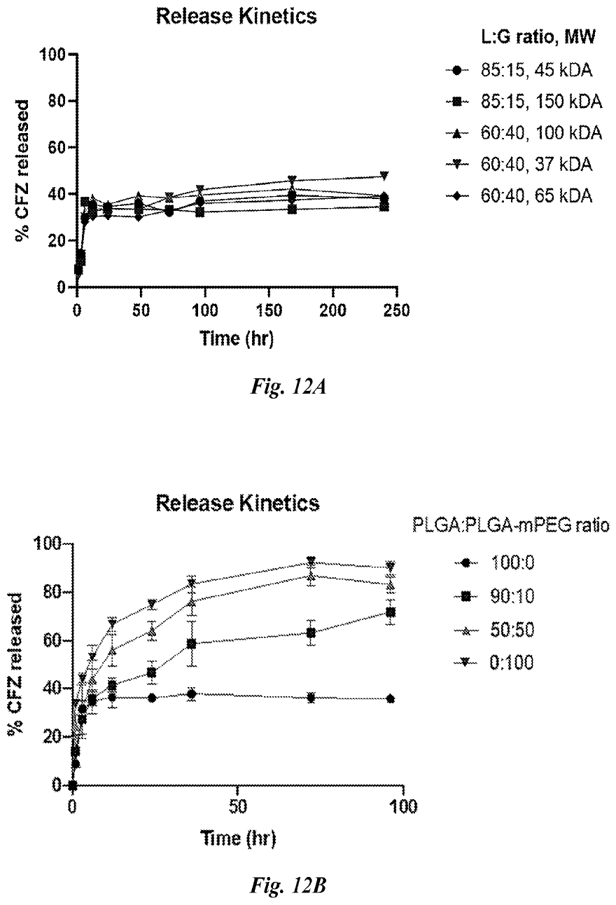

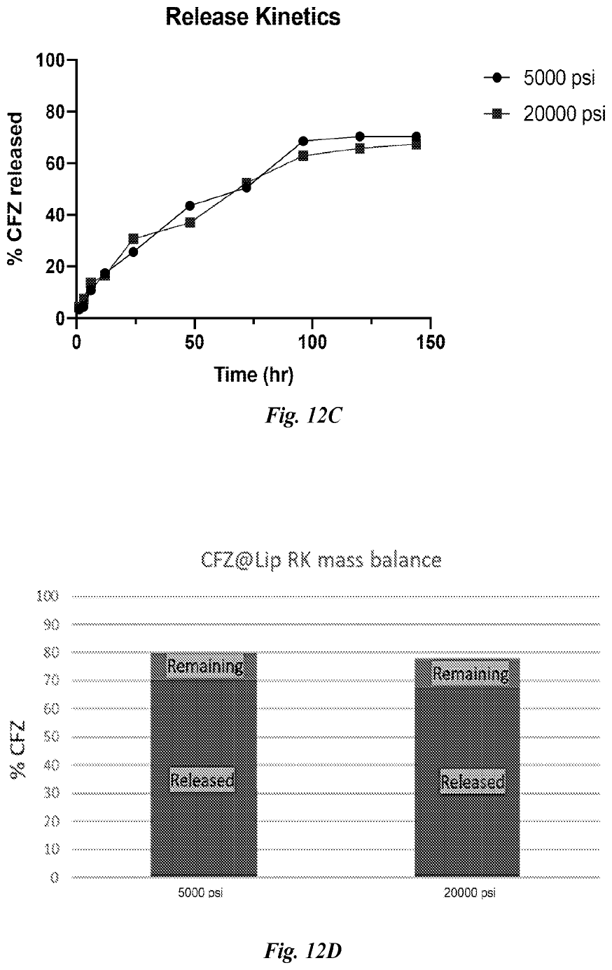

The release of CFZ in the produced (uncoated) nanoparticles (CFZ@NP) was prepared at a concentration equivalent to CFZ at 3 μg/mL was measured in 15 mL 0.2% Tween80/PBS under the sink condition (sink condition—the maximum solubility of CFZ in 0.2% PBS solution with a low-concentration detergent solution (PBST): 9 μg/mL), and the concentration of CFZ in the release medium was measured at different time points (FIG. 12A). 200 μL of samples were collected at each time point indicated in FIG. 12A, then centrifuged for 20 minutes @16000 rcf. 150 μL of supernatants were collected, then analyzed. 150 μL of 0.2% PBST was added to the collected sample, then added back to the original sample. Regardless of the formulation, no significant change in the release was observed.

### Alternation of PLGA Composition:

Another approach is to improve the release was by utilizing polyethylene glycol (PEG) conjugated PLGA, which can increase the porosity of the polymer by forming a water channel. The wetting ability of PEG may promote the diffusion and release of drugs. A previous study has shown that incorporation of PEG to PLGA polymer has improved the release of the doxetaxel (see Rafiei and Haddadi, Docetaxel-loaded PLGA and PLGA-PEG nanoparticles for intravenous application: pharmacokinetics and biodistribution profile, Int J Nanomedicine 12: 935-947 (2017)). Therefore, PEG conjugated PLGA was assessed in an effort to improve the release of the encapsulated CFZ, by replacing PLGA with PEG-conjugated PLGA to varying degrees.

Briefly, different ratios of PLGA to PLGA-methoxy-polyethylene glycol (mPEG) were mixed to encapsulate CFZ. More specifically, PLGA (LA:GA=85:15, 97 kDA) was mixed with PLGA-mPEG (MW ˜5,000:55,000 Da LA:GA=50:50) in 1 mL dichloromethane (DCM) to replace 10%, 50%, or 100% of PLGA. 5 mg CFZ was added to 1 mL of PLGA/PLGA-mPEG dissolved DCM (at 10% weight ratio).

The CFZ+PLGA/PLGA-mPEG solution was added to 5 mL of 4% PVA. The mixture was emulsified by sonication for 2 minutes (2 seconds on, 2 seconds off, 40% amplitude). The emulsification was added into 25 mL of DI water. The solution was stirred for 3 hours, then rotavap for additional 30 minutes at 20 mbar. The PLGA/PLGA-mPEG-NPs loaded with CFZ were collected using centrifugation for 20 minutes, 43400 rcf. The PLGA/PLGA-mPEG-NPs loaded with CFZ were then washed with DI water, sonicated (10 seconds, 20% amplitude), and centrifuged for 15 minutes, 43400 rcf, twice. The pellets were collected and stored at 4° C. Replacement of PEG did not affect the physical characteristics of the particles (Table 3).

TABLE 3
CFZ loaded PEG-conjugated PLGA nanoparticle characterization

| | | | Drug |
|---|---|---|---|
| Sample (PLGA:PLGA-PEG | Size | ZP | loading |
| ratio) | (d · nm) | (mV) | (wt %) |
| CFZ@PLGA/PLGA-PEG | 217 ± 14 | −0.56 ± 1.0 | 5.58 ± 0.17 |
| (100:0) | | | |
| CFZ@PLGA/PLGA-PEG | 229 ± 5 | −0.64 ± 0.44 | 5.84 ± 0.32 |
| (90:10) | | | |
| CFZ@PLGA/PLGA-PEG | 230 ± 7 | 0.017 ± 0.094 | 5.73 ± 0.4 |
| (50:50) | | | |
| CFZ@PLGA/PLGA-PEG | 232 ± 5 | −0.32 ± 0.68 | 5.68 ± 0.32 |
| (0:100) | | | |

As noted above, the release of CFZ in the produced nanoparticles were measured in 0.2% Tween80/PBS under the sink condition. Samples were taken out at different time points and the amount of the CFZ release was measured (FIG. 12B). As expected, the more PLGA was replaced with PEG-conjugated PLGA, the more CFZ release was observed.

### Alternative Delivery Vehicle:

Alternative approaches for enhancing the release of CFZ were also explored by encapsulating CFZ in liposomes, instead of PLGA. Liposomes have shown to provide sustained release of PTX (see. e.g., Liu et al., Mixed Liposome Approach for Ratiometric and Sequential Delivery of Paclitaxel and Gemcitabine, AAPS PharmSciTech, 19: 693-699 (2018)). Therefore, liposomes were screened for the delivery of CFZ.

Liposomes containing CFZ were produced with a high-pressure homogenizer at 5,000 psi or 20,000 psi (Table 4).

TABLE 4
CFZ loaded liposome characterization

| | Size | ZP | Drug loading |
|---|---|---|---|
| Sample | (d · nm) | (mV) | (wt %) |
| CFZ@Lip - 5,000 psi | 110 | 0.7 | 1.1% |
| CFZ@Lip - 20,000 psi | 107 | −2.5 | 1.2% |

Briefly, DPPCs were dissolved in a mixture of 3 mL chloroform. CFZ was added to this solution at 2.1 wt % of the lipid, and the CFZ/lipid solution was transferred to a round-bottom flask and rotated with a rotary evaporator under vacuum at 45° C. for 30 minutes to remove the solvent and form a thin lipid film. The liposome suspension was briefly sonicated with an ultrasonic probe. The liposomes were extruded through high pressure homogenizer at 5,000 to 20,000 psi. Liposomes were collected via centrifugation at 175,000 rcf×20 minutes. Liposomes loaded with CFZ were prepared at a concentration equivalent to CFZ at 3 μg/mL (sink condition—maximum solubility of CFZ in 0.2% PBST: 9 μg/mL) in 15 mL 0.2% Tween 80/PBS. 200 μL of samples were collected at each time point indicated in FIG. 12C and centrifuged for 20 minutes @175,000 rcf. 150 μL of supernatants were collected, then analyzed. 150 μL of 0.2% PBST was added to the collected sample, then added back to the original sample.

Regardless of the pressure used to produce the nanoparticles, the nanoparticles demonstrated similar size and drug loading. The release of CFZ was tested in the similar way as other studies. The liposome formulations, irrespective of the pressure used for the production, demonstrated similar release profiles over time (FIG. 12C) with 70% of the loaded CFZ being released over 144 hours.

FIG. 12D shows liposome encapsulated CFZ release kinetics mass balance, indicating that the majority of the encapsulated CFZ was released in 6 days (e.g., linearly released over time).

### Example 10

### Cytotoxicity of PTX or CFZ Encapsulated PLGA Nanoparticles

The cytotoxicity of PTX and CFZ was tested against CT26 murine carcinoma and JAWSII DCs. As the nanoparticles hereof can be used to deliver ICD inducers to create tumor antigens, which need to be taken up by APCs, in certain embodiments, it is important for the formulations to kill tumor cells, while saving the host immune cells.

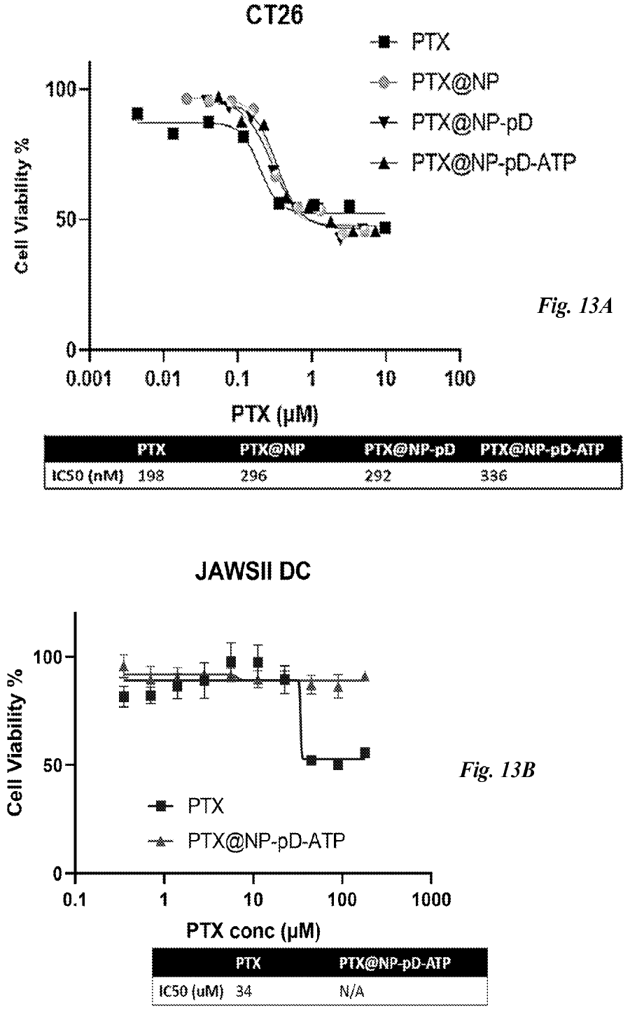

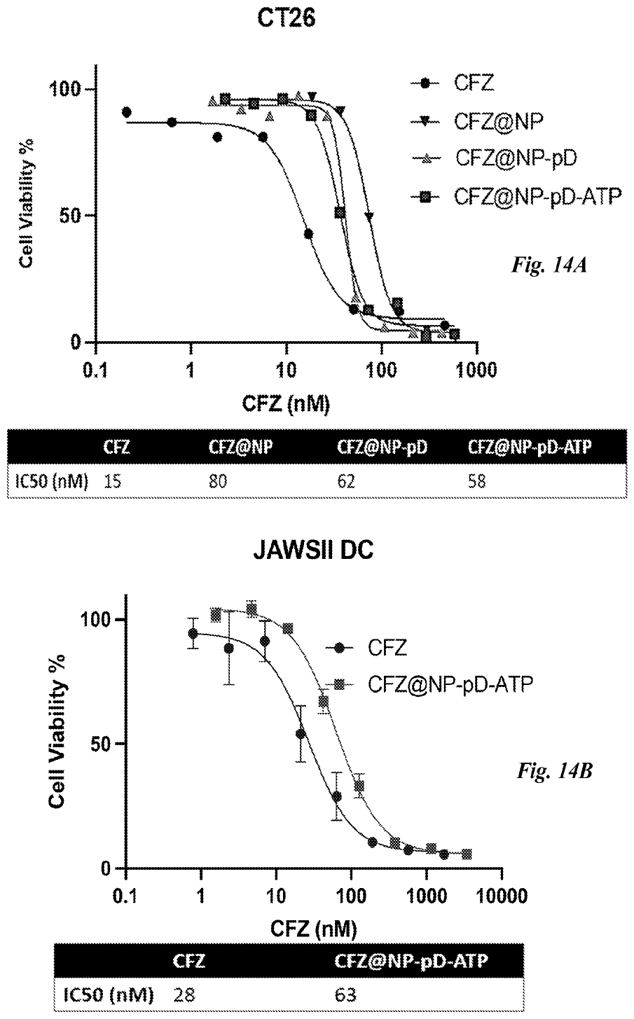

Cytotoxicity was assessed by the MTT assay. CT26 cells were seeded in 96 wells with 3,000 cells/well and cultured until 30% confluency. Cells were treated with varying concentrations of Drug, Drug@NP, Drug@NP-pD, Drug@NP-pD-ATP for 24 hours. After 24 hours, media was replaced, the cells were incubated for an additional 24 hours, and an MTT assay was performed to measure the number of viable cells present (see FIGS. 13A and 14A).

For JAWSII cells, JAWSII cells were seeded in 96 wells with 20,000 cells/well. Cells were treated with varying concentration of PTX for 96 hours, and an MTT assay was performed to measure the number of viable cells present (see FIGS. 13B and 14B).

PTX nanoparticle formulations showed cytotoxicity against CT26 tumor cells, with minimal toxicity against JAWSII DCs (FIGS. 13A-13B). PTX is generally better tolerated by immune cells than cancer cells as PTX, a cytostatic chemotherapy, does not affect cells that do not proliferate as rapidly as tumor cells, such as DCs. We suspect that the sudden decrease of DC viability at 30 μM or above may have to do with crystallization of free PTX, which could have induced physical damages to the cell membrane (cancer cells have not been exposed to 30 μM or higher due to the high sensitivity).

CFZ nanoparticle formulations were toxic to both CT26 tumor cells and JAWSII dendritic cells (FIGS. 14A-14B). Such difference is likely due to the difference in mechanism of action: CFZ is a proteasome inhibitor, which may prevent degradation of pro-apoptotic factors such as the p53 protein, permitting activation of programmed cell death, regardless of the cell type. From these findings, PTX was chosen as the lead candidate for future studies to minimize the potential for immunotoxicities.

### Example 11

### Tumor Processing for Immune Cell Profiling

The following protocol was used for processing tumors for the in vivo studies described herein.

Tumors were harvested and minced into small pieces and digested by medium containing 1.5 mg/mL collagenase type 4, 0.2 mg/mL DNase I and 0.2 mg/mL hyaluronidase at 37° C. for 2 hours. Cell suspension was filtered through 100 μm followed by 40 μm cell strainers. Cells were centrifuged at 500 g for 8 minutes. Pellets were resuspended in 5 mL of ACK lysing buffer, then incubated for 5 minutes. 50 mL PBS were added to stop the lysing and centrifuged at 500 g for 8 minutes. Cells were resuspended in the staining buffer, which was a buffered saline solution containing FBS and sodium azide (0.09%) as a preservative. Cells were stained with anti-mouse CD16/32 antibody for 10 minutes at 4° C. to block non-specific binding of immunoglobulin to Fc receptors, followed by staining antibodies for 20-30 minutes at 4° C. and centrifuged at 500 g for 8 minutes.

Cells were fixed with 1 mL of 4% PFA at room temperature for 20 minutes and, thereafter, centrifuged at 500 g for 8 minutes. Cells were collected and suspended in 300 μL of PBS at 4° C. for flow cytometer.

### Example 12

### NP-pD-ATP Recruits DCs to the Tumor

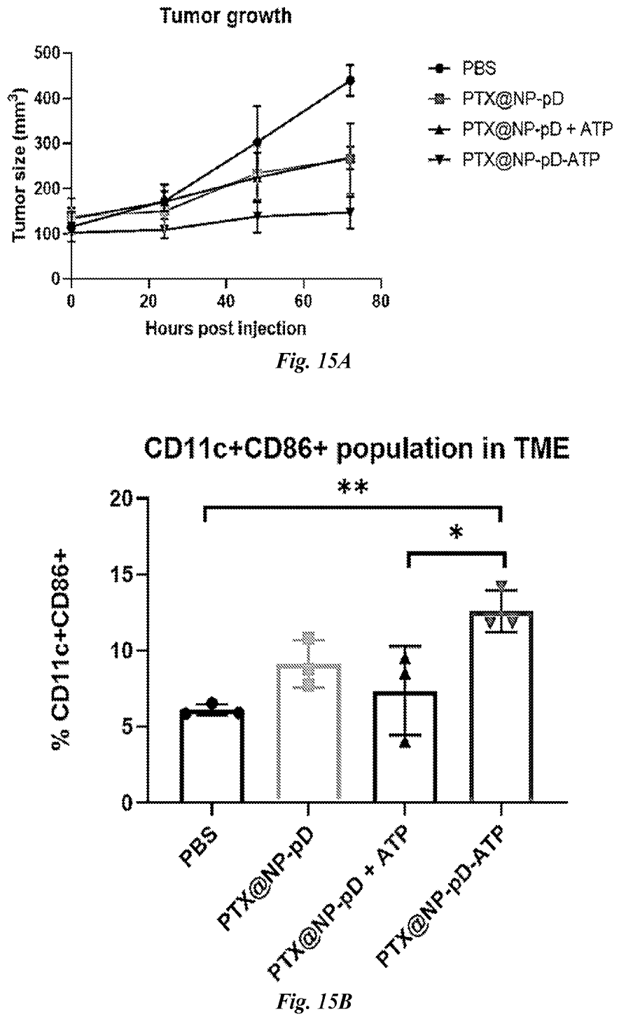

APCs can initiate anti-tumor immune response by presenting tumor antigens produced by ICD inducers to the T cells. To test whether NP-pD-ATP can attract APCs, as suggested by the above-described in vitro Transwell study, PTX@NP-pD-ATP was injected intratumorally to the CT26 tumor bearing mice, and the number of DCs within the tumor microenvironment (TME) and tumor growth were analyzed 3 days after the injection (FIGS. 15A and 15B).

More specifically, female BALB/c mice (4-6 weeks-old) were inoculated with 3×10⁵ CT26 cells subcutaneously in the upper flank of right hind limb. When tumors grew to about 50-100 mm³, the mice were randomly assigned to 3 groups (n=3 per group) and treated with an intratumoral injection of 100 μL of PBS. PTX@NP-PD, or PTX@NP+ATP mixture. Tumor growth was monitored and, after 72 hours of injection, tumors were collected to evaluate the number of dendritic cells in the tumor. Tumors were prepared into a single cell suspension (as described in Example 11) and stained with antibodies for immunophenotyping. Percentages of DCs (CD45⁺CD86⁺CD11c⁺) were measured by flow cytometry.

Tumor volumes were best suppressed with PTX@NP-pD-ATP by 3 days, as compared to PBS, PTX@NP-pD, or the mixture of PTX@NP-pD with soluble ATP. PTX@NP-pD-ATP significantly increased the number of DCs within the tumor when compared to the other groups. The addition of soluble ATP did not increase DC infiltration into the tumor, but rather decreased it as compared to the PTX@NP-pD-ATP. Such effect may be driven by the instability of ATP due to the presence of degrading enzymes (CD73 and CD39) commonly expressed on the surface of the tumor cells. Such degrading enzymes can convert ATP to AMP or adenosine, which can induce immunosuppressive environment that can hinder DC recruitment.

Unlike the mixture of PTX@NP-pD and free ATP, PTX@NP-pD-ATP helped recruit DCs to the TME upon local injection in the TME (FIG. 15B) and suppressed the tumor growth for the first 3 days (FIG. 15A). This supports that NP-pD-ATP can help to preserve stability of ATP and increase the infiltration of DCs into the tumor, thereby inducing antitumor immunity.

### Example 13

### Nanoparticle Formulation Enhances the Retention of Nanoparticles in Tumor

To test if ATP-bound nanoparticles extend the retention of ATP in the tumor, tumor bearing mice were treated with fluorescent dye-bound nanoparticle and imaged over time. While it is possible to simply measure the amount of ATP in the tumor after injection of ATP or NP-pD-ATP, it will be difficult to distinguish between exogenous ATP (e.g., from nanoparticles) and endogenous ATP (e.g., derived from the cells) as ATP is a common energy source in the body. Further, ATP from the injection may degrade into ADP or AMP. Accordingly, amine-terminated dye was used in lieu of ATP to simulate the behavior of soluble ATP and nanoparticle-bound ATP. More specifically, amine conjugated Cy7 dye was used as its water solubility is similar to that of ATP and the florescent spectrum of amine conjugated Cy7 dye does not overlap with the auto florescence from the mice body.

Cy7 coated nanoparticles (NP-pD-Cy7) were produced in the same manner as NP-pD-ATP except for the wt %. PLGA nanoparticles were incubated in dopamine HCl solution in bicine buffer (10 mM, pH 8.5) for 1 hour at a dopamine HCl-to-NP weight ratio of 0.5/1. When nanoparticles manifested the dark color of polymerized dopamine, nanoparticles were collected by centrifugation (20 minutes×25000 rpm) and washed twice with DI water to remove excess dopamine and pD.

20 mg PLGA-pD were incubated with 50 μg of Cy7-amine (0.25% Cy7-amine to nanoparticle weight ratio) for 1 hour in 5 mL bicine buffer (10 mM, pH 8.5). The nanoparticles were collected by centrifugation at 25000 rpm for 20 min at 4 C and washed twice with DI water.

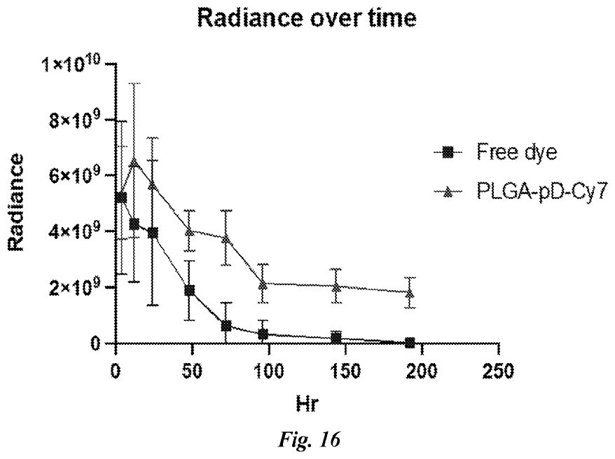

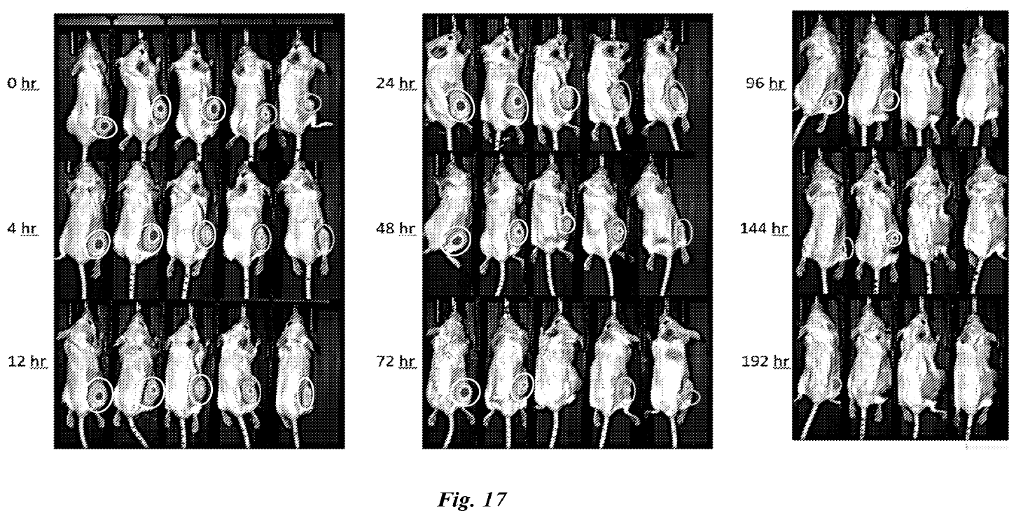

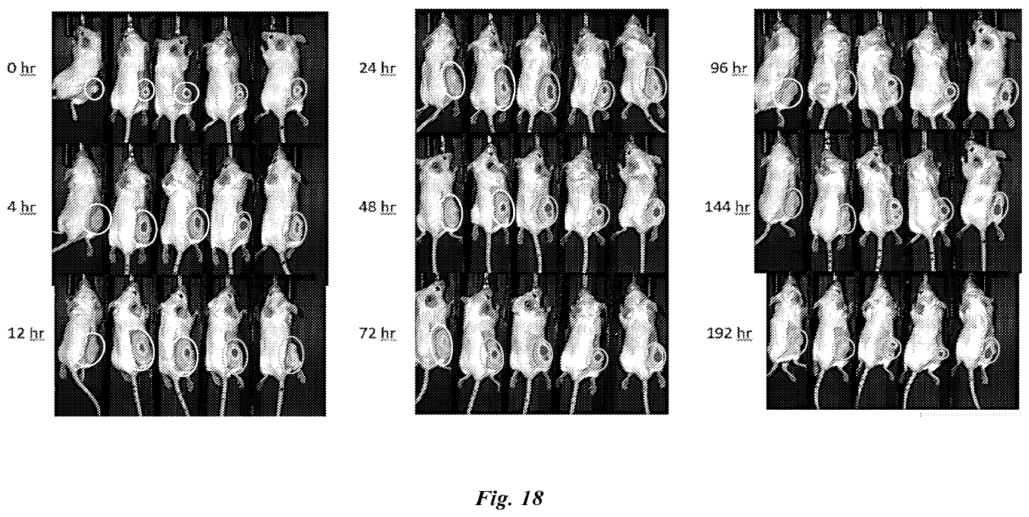

The resulting NP-pD-Cy7 was injected intratumorally to the CT26 bearing mice, and the florescence intensity of Cy7 was measured over time with the AMI imager (FIG. 16). In contrast, the Cy 7 signal from the free Cy7 diminished quickly and started to be non-detectable 3 days after the injection (FIGS. 16 and 17). On other hand, signals of Cy7-conjugated nanoparticles persisted even after 8.2 days (FIGS. 16 and 18), suggesting that ATP stably bound to the nanoparticles can remain longer within the tumor as compared to soluble ATP.

### Example 14

### Antitumor Efficacy of PTX Encapsulated NP-pD-ATP by Intravenous Delivery

Systemic antitumor efficacy of PTX@NP-pD-ATP was assessed various models of tumor bearing mice (CT26 syngeneic model and B16F10 syngeneic model). The mice were treated with PTX equivalent of 20 mg/kg intravenously 4 times every 3 days once the tumor reached the volume of 50 mm³. Tumor growth was observed every daily.

### CT26 Syngeneic Model

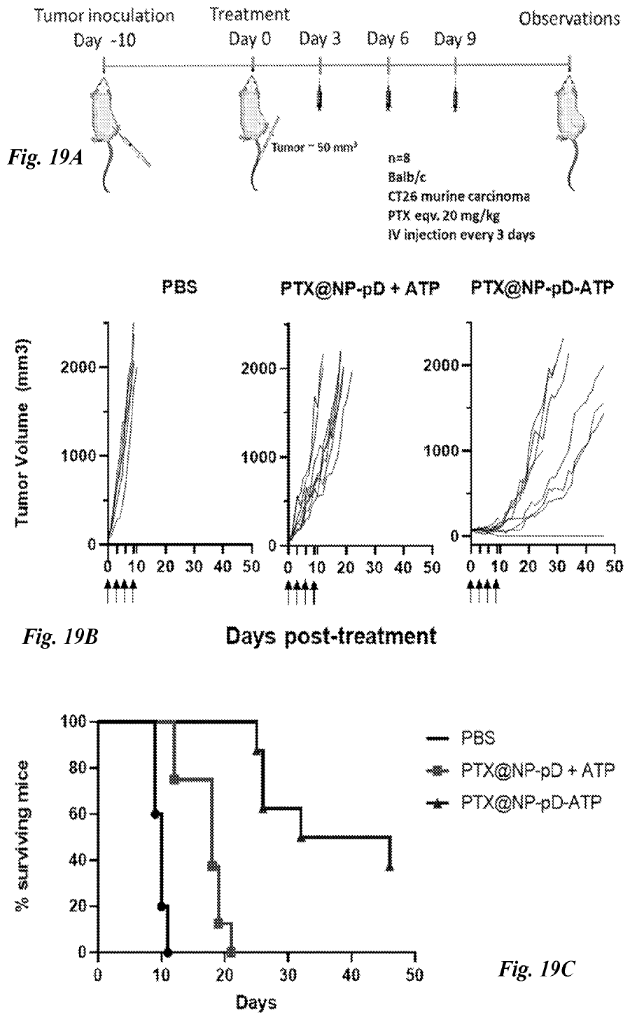

Female BALB/c mice (4-6 weeks-old) were inoculated with 3×10⁵ CT26 cells subcutaneously in the upper flank of right hind limb (FIG. 19A). When tumors grew to about 50-100 mm³, the mice were randomly assigned to 3 groups and treated with an intravenous injection of 150 μL of PBS, PTX@NP-pD, or PTX@NP+ATP mixture, and PTX@NP-pD-ATP every 3 days, 4 times total. The PTX dose was 20 mg/kg equivalent for all the PTX receiving group. Tumor growth was monitored.

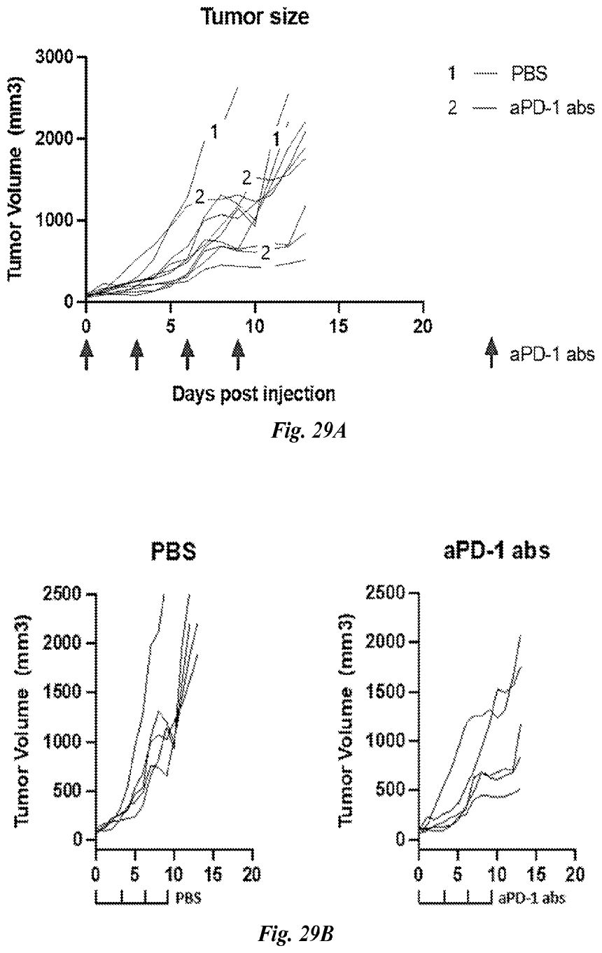

PTX@NP-pD-ATP demonstrated better antitumor efficacy as compared to treatment with PBS or the mixture group, yielding complete regression in 1 mouse out of 5 mice (FIGS. 19B and 29C). However, the tumor still grew in most of the mice. Tumors were initially suppressed while the treatments were given, but once the treatments were stopped after 4 times, tumors started to regrow.

### B16F10 Syngeneic Model

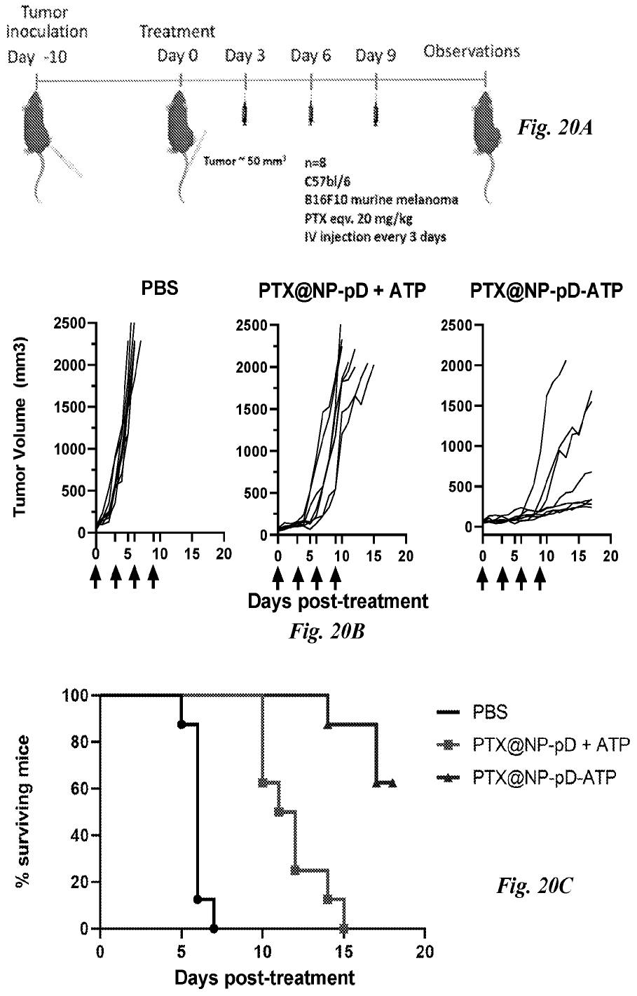

Male C57BL/6 mice (4-6 weeks-old) were inoculated with 5×10⁵ B16F10 cells subcutaneously in the upper flank of right hind limb (FIG. 20A). When tumors grew to about 50-100 mm³, the mice were randomly assigned to 3 groups and treated with an intravenous injection of 150 μL of PBS, PTX@NP-PD, PTX@NP+ATP mixture, and PTX@NP-pD-ATP every 3 days, 4 times total. The PTX dose was 20 mg/kg equivalent for all the PTX receiving group. Tumor growth was monitored. FIG. 20B shows a graph of tumor size following intravenous injection of each group. FIG. 20C shows a graph of the percent survival of the mice after the treatments (or lack thereof).

Consistent with the previous observation in the CT26 syngeneic model, the PTX@NP-pD-ATP group demonstrated superior antitumor efficacy as compared to the PBS and PTX@NP-pD+ATP groups. However, the tumor growth of B16F10 tumors were faster than that of CT26 tumors, possibly owing to the smaller number of immune cells in the tumor and quicker doubling time.

Nonetheless, the effectiveness of the PTX@NP-pD-ATP formulation was consistent across the different tumor models, supporting the enhanced efficacy of ATP when conjugated to the surface of nanoparticle. This enhanced efficacy could result from the increased stability and retention of ATP from the coating and nanoparticle formulation, which leads to the recruitment of nearby immune cells and elicits a desired immune response.

### Example 15

### Antitumor Efficacy of PTX Encapsulated NP-pD-ATP in CT26 Model by Intratumoral Delivery

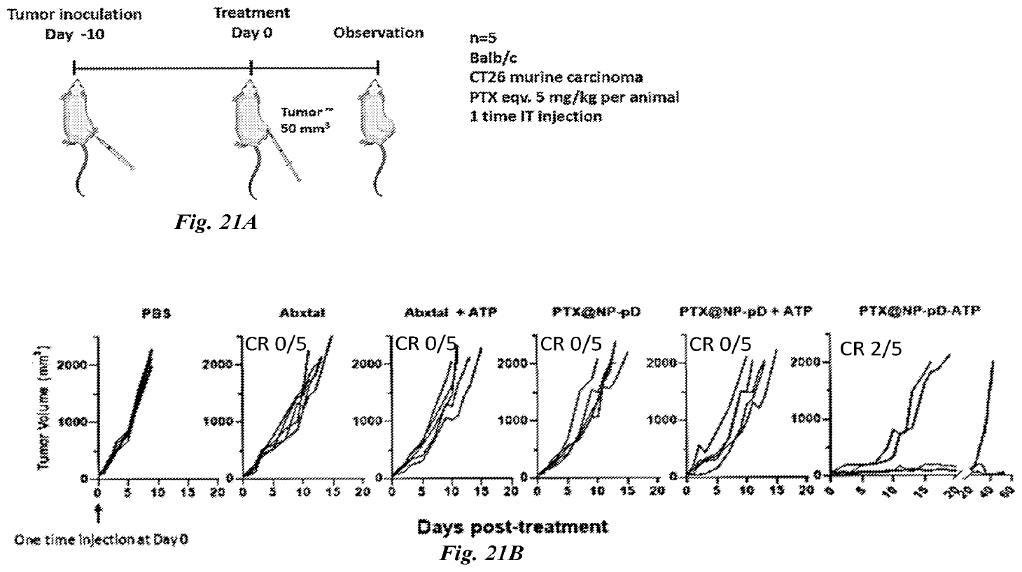

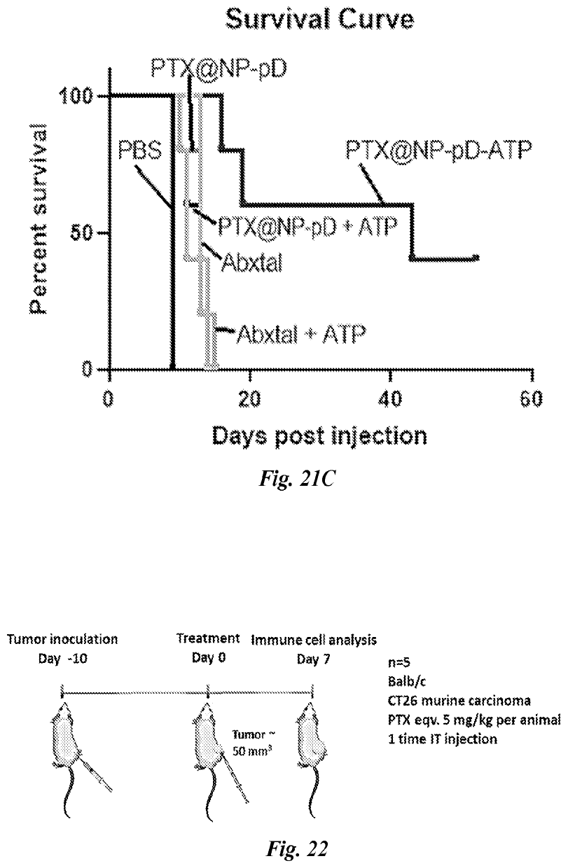

With intravenous injection, there is a chance that a different amount of PTX may be delivered to the tumor as compared to the amount injected. To test if ATP-bound nanoparticle increases the antitumor effect fixing PTX delivery to tumor, another set of animal studies was performed, where mice were treated intratumorally. Unlike intravenous injections, the intratumoral injections were given only once directly into the tumor, and the tumor growth was monitored (FIG. 21A). As the treatments were delivered intratumorally, the PTX amount delivered to the tumor was expected to be the same between the groups. Additional control groups were also included in the study and Abxtal, a PTX carrier free nanocrystal formulation, was used in place of Abraxane (a current standard of PTX therapy) because Abxtal is as effective as Abraxane. (See Park and Yeo, Albumin-coated nanocrystals for carrier-free delivery of paclitaxel, J. Controlled Release 263: 90-101 (2017); see also Park et al., A Comparative In Vivo Study of Albumin-Coated Paclitaxel Nanocrystals and Abraxane, Small 14: e1703670 (2018)).

Female BALB/c mice (4-6 weeks-old) were inoculated with 3×10⁵ CT26 cells subcutaneously in the upper flank of right hind limb. Tumor growth was monitored and, when tumors grew to about 50-100 mm³, the mice were randomly assigned to 5 groups and treated with an intratumoral injection of 100 μL of PBS. PTX (Abxtal). PTX (Abxtal)+ATP. PTX@NP-pD. PTX@PLGA-pD+ATP, and PTX@PLGA-pD-ATP. The PTX dose was 5 mg/kg equivalent for all the PTX receiving groups. Tumor growth was again monitored.

Among the treatment groups. PTX@NP-pD-ATP demonstrated the best antitumor efficacy, resulting in the complete remission in 2 out of 5 mice (FIGS. 21B and 21C). The additional delivery of soluble ATP along with either Abxtal or PTX@NP-pD did not suppress the tumor significantly, as demonstrated by the tumor growth curve and the survival curve (FIGS. 21B and 21C). This supports that NP-pD-ATP can enhance the retention of ATP, as previously seen in the Cy7 study (Example 13), thereby increasing the infiltration of immune cells to the tumor.

### Example 16

Immune Cell Profiling of CT26 Tumors after Intratumoral Delivery of PTX Loaded NP-pD-ATP

To better understand the changes of immune cells in the TME, an immune cell analysis was performed after 7 days of intratumoral injection (FIG. 22). 7 days was determined as an appropriate time to study the number of immune cells, as both innate and adaptive immune systems would be active by this time.

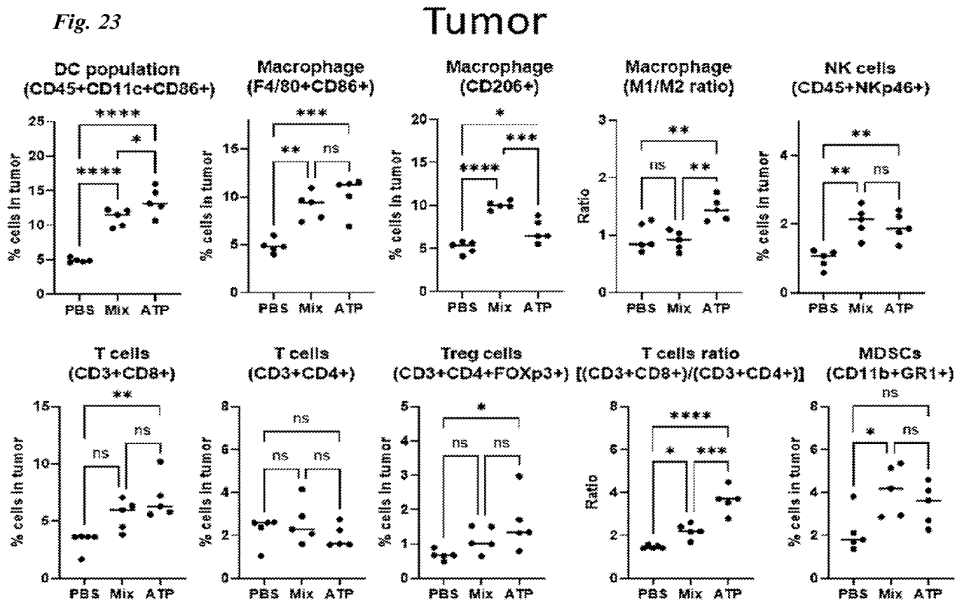

Female BALB/c mice (4-6 weeks-old) were inoculated with 3×10⁵ CT26 cells subcutaneously in the upper flank of right hind limb. When tumors grew to about 50-100 mm³, the mice were randomly assigned to 3 groups and treated intratumorally with an injection of 100 μL of PBS. PTX@PLGA-pD+ATP, or PTX@PLGA-pD-ATP. The PTX dose was 5 mg/kg equivalent for all of the PTX receiving groups. 7 days or 20 days after the injection, tumors, spleen, and tumor draining lymph nodes were harvested and the number of immune cells were measured by flow cytometry (FIGS. 23-25). As Abxtal. Abxtal+ATP. PTX@NP-pD, and PTX@NP-pD+ATP showed similar antitumor efficacy with each other (see Example 15). PTX@NP-pD+ATP was chosen as a representative control. Therefore, only three treatment groups were compared (PBS. PTX@NP-pD+ATP and PTX@NP-pD-ATP).

In the TME (FIG. 23), the number of DCs in mice treated with PTX@NP-pD-ATP was significantly higher than those of the PTX@NP-pD+ATP (mixture) treatment group or PBS control group. The number of M1 macrophage (anti-tumor like macrophage) increased in both the PTX@NP-pD-ATP and mixture groups when compared to PBS (control group): however, the mixture group also had an increase of M2 macrophage (pro-tumor like macrophage). The M1/M2 ratio in the tumor was increased only in PTX@NP-pD-ATP treatment group. Natural killer (NK) cells increased in both the PTX@NP-pD-ATP and mixture treatment groups as compared to PBS (control group). Overall, the innate immune cell population significantly increased with the treatment of PTX: however, the addition of soluble ATP did not further increase the immune cell population, while the use of ATP conjugated to the nanoparticle did.

PTX@NP-pD-ATP significantly increased the amount of CD8+ T cells within in the tumor compared to mixture or PBS. No significant increase in the CD4+ T cells were observed. However. PTX@NP-pD-ATP treatment increased the amount of Treg cells in the tumor as compared to the PBS (control) group. Such effect is likely due to the continuous inflammation caused by DCs and CD8+ T cells in the tumor, which promotes the recruitment of Treg cells to counteract the activity of dendritic cells and CD8+ T cells. Both the PTX@NP-pD-ATP and mixture treatment groups exhibited apparent an increase in myeloid-derived suppressor cell (MDSC) population, but only the mixture treatment group showed a significant difference compared to the control group.

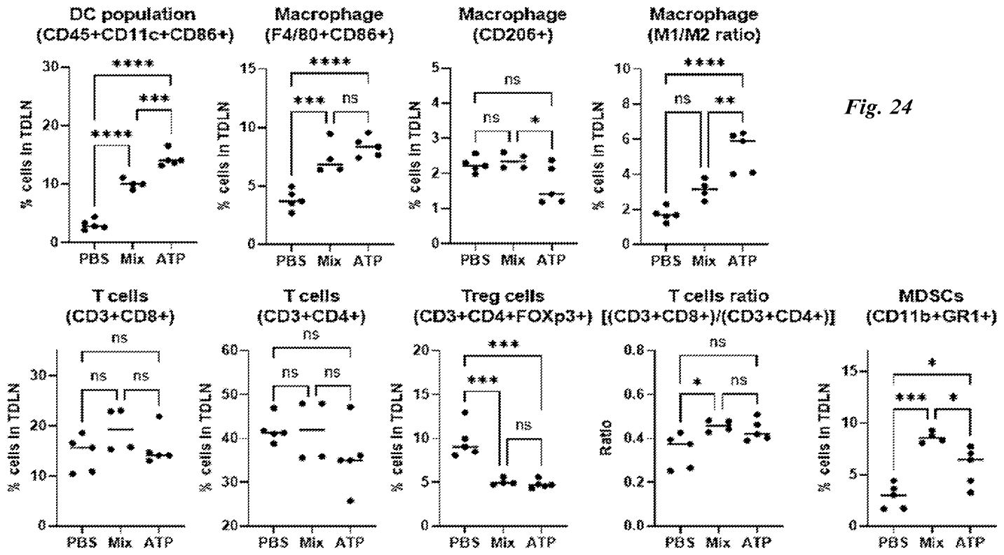

Tumor draining lymph nodes (TDLN), an immune organ next to the tumor, were also analyzed in the similar manner as the tumor. As shown in FIG. 24, significant DC and M1 macrophage populations were observed in the mixture and PTX@NP-pD-ATP treatment groups (with a greater increase observed in the PTX@NP-pD-ATP treatment group). Overall, the innate immune cell trend in TDLN mirrored that of the tumor. However. T cells demonstrated no significant trend. Additionally, the population of Treg cells in the TDLN decreased with the treatment groups (PTX@NP-pD-ATP or mixture). Both PTX@NP-pD-ATP and mixture treatment groups showed significant increases of MDSCs in the TDLN: however, the PTX@NP-pD-ATP treatment group had a significantly lower number of MDSCs in the TDLN as compared to the mixture treatment.

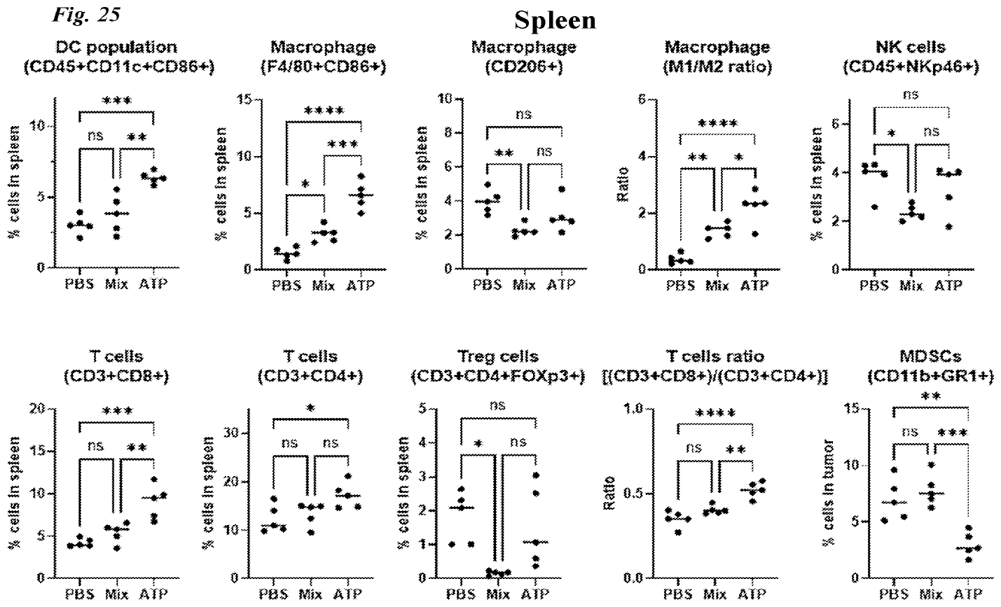

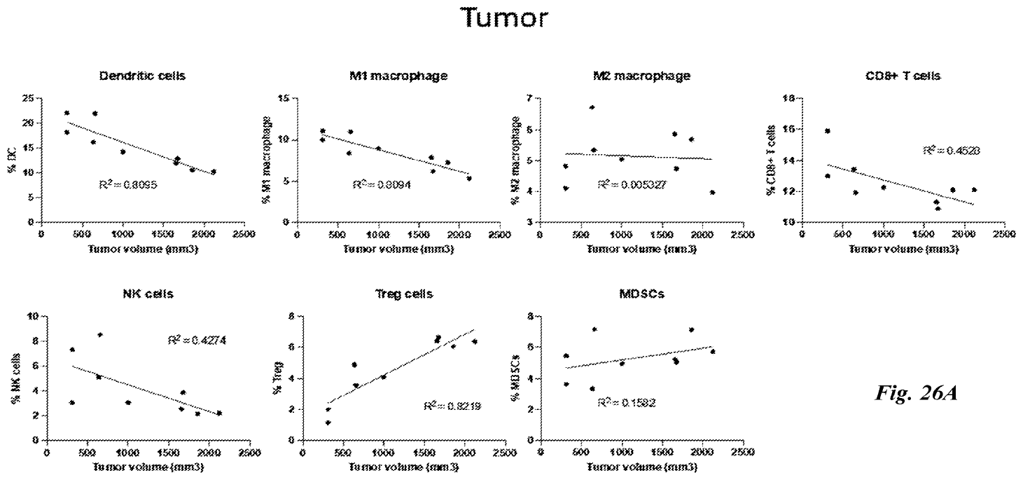

The population of immune cells in the spleen were also analyzed (FIG. 25). The spleen has various of types of immune cells (1.5% DCs. 5% of macrophages. 23% of T cells. 1.2% of Tregs. 64% of B cells) and regulates the development of innate and adaptive immunity. Both the DC and M1 macrophage population increased in PTX@NP-pD-ATP or mixture treatment groups as compared to PBS (control) group, with the extent of increase significantly higher in the PTX@NP-pD-ATP treatment group as compared to the mixture treatment group. A significant increase of CD8+ T cells was observed only in the PTX@NP-pD-ATP treatment group, along with a slight increase of CD4+ T cells. Treg cell population could not be compared. MDSC population significantly decreased with the treatment of PTX@NP-pD-ATP, contrary to the observation in the tumor.

Overall. DC population, regardless of the sampling location (i.e., tumor. TDLN, spleen), demonstrated a consistent increase with the treatment of PTX@NP-pD-ATP or the mixture. However, the extent of increase observed was higher with the PTX@NP-pD-ATP treatment than the mixture treatment. Also, the M1 population and the M1/M2 macrophage ratio were significantly higher in the PTX@NP-pD-ATP treatment group, than the PBS control group or the mixture treatment group. This supports that ATP conjugation of nanoparticle enhanced the recruitment of DCs and macrophages, with such enhancement of innate immune cell recruitment translating into stronger activation of adaptive immune cells as demonstrated by higher CD8+ T cell population in tumor and spleen. The increase of Tregs and MDSCs observed in the tumor and TDLN could potentially account for the growth of the tumor in the later phase.

### Example 17

Changes of the Immune Cell Population in the CT26 Tumor with Respect to Tumor Size after Intratumoral Injection

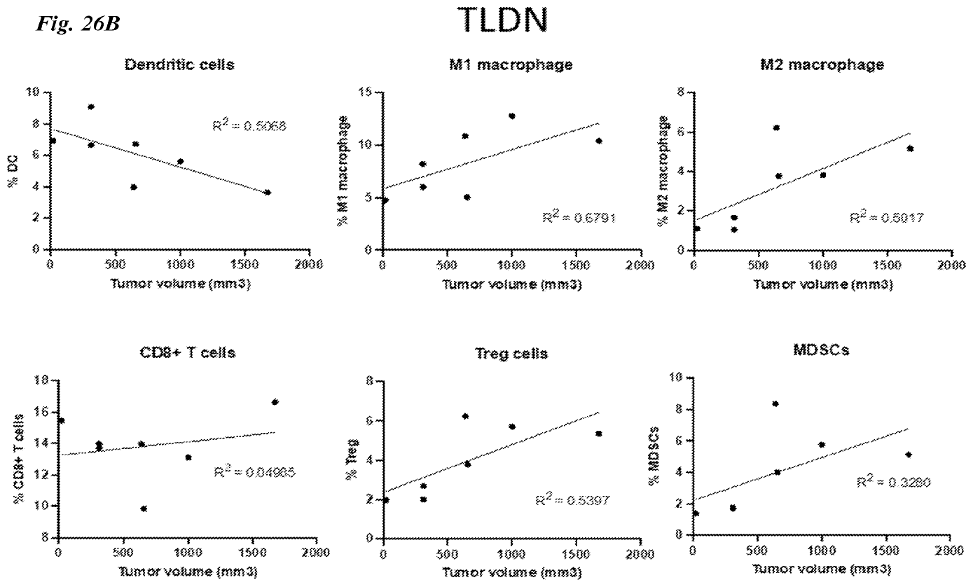

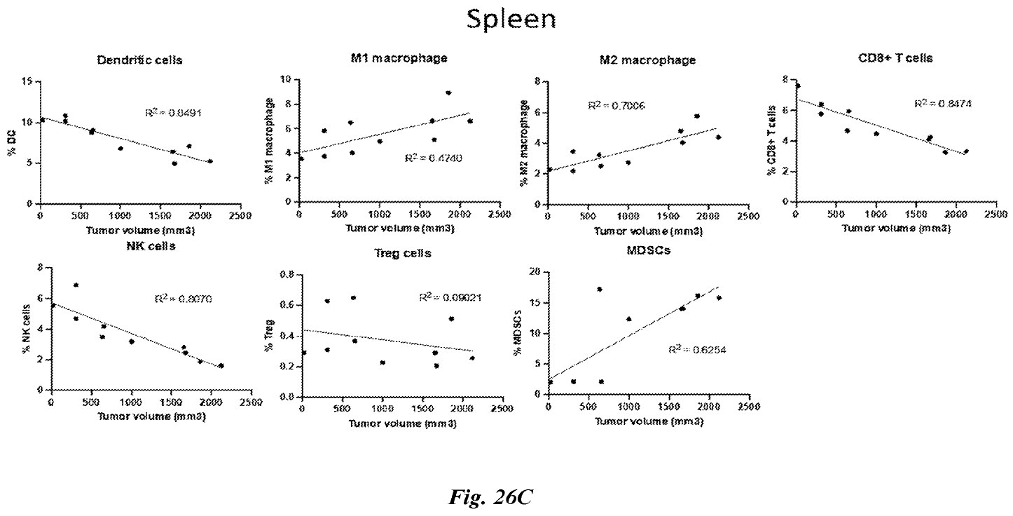

While PTX@NP-pD-ATP suppressed tumor growth better than the mixture treatment group (PTX@NP-pD+ATP), the tumor grew after the initial suppression in varying degrees. To investigate the potential role of immune cells in the later relapse of tumor, CT26 tumors were treated with PTX@NP-pD-ATP intratumorally, followed by analysis of the immune cells from the tumor (FIG. 26A), lymph nodes (FIG. 26B), and spleens (FIG. 26C) 25 days after the intratumoral injection (performed as previously described in the aforementioned studies).

The number of anti-tumor immune cells highly correlated with the size of the tumor. As the tumor grew; the Treg population increased along with the population of MDSCs, and the number of anti-tumor immune cells (e.g., DCs, macrophages, and T cells) decreased. Although the spleen and TDLN did not always show the same results as tumor (for example, M1 macrophage population reduced in tumor, but increased in the spleen and TLDN), the overall trend supports that the lack of anti-tumor immune cells is responsible for the late relapse of tumor.

### Example 18

Combination Therapies with Anti-PD-1 Antibodies and PTX@NP-pD-ATP

The increase of immunosuppressive cells in the tumor observed in Example 17 was expected, as tumors have feedback mechanisms to counteract the activity of T cells. While an increased number of T cells in the tumor was observed in the previous studies, IFN gamma released from the T cells can induce the upregulation of PD-L1 expressions in the tumor, which can result in exhaustion of CD8 T cells and recruitment of Tregs. See. e.g., Jorgovanovic et al., Roles of IFN-gamma in tumor progression and regression: a review, Biomark Res 8: 49 (2020). The recruited Tregs can downregulate the activation of tumor residing CD8 T cells and DCs, further mitigating the activity of adaptive immunity.

As the presence of Tregs has been highly correlated with poor outcomes of cancer immunotherapy, therapeutics have been developed to prevent the proliferation of Tregs or to prohibit the interaction of Tregs with other immune cells. See Han et al., Turning the Tide Against Regulatory T Cells, Front Oncol 9: 279 (2019). While the mechanism of action for each strategy is different from each other, three strategies can be used to prevent the activation of or to deplete Treg cells: (i) anti-cancer drugs. (ii) Treg cell-depleting agents, and/or (iii) the conversion of Treg cells to immune stimulating cells. Han (2019), supra.

Out of possible therapeutic options to downregulate Treg cells, anti-PD-1 antibodies were identified as an additional therapeutic agent to study further. This is because anti-PD-1 antibodies can relieve the suppression of cytotoxic CD8 T cells and downregulate Tregs, while preventing the interaction of T cells along with other immunosuppressive cells. See Yoshida et al., Anti-PD-1 antibody decreases tumour-infiltrating regulatory T cells. BMC Cancer 20: 25 (2020) and Ravelli et al., Immune-related strategies driving immunotherapy in breast cancer treatment: a real clinical opportunity. Expert Rev Anticancer Ther 15: 689-702 (2015).

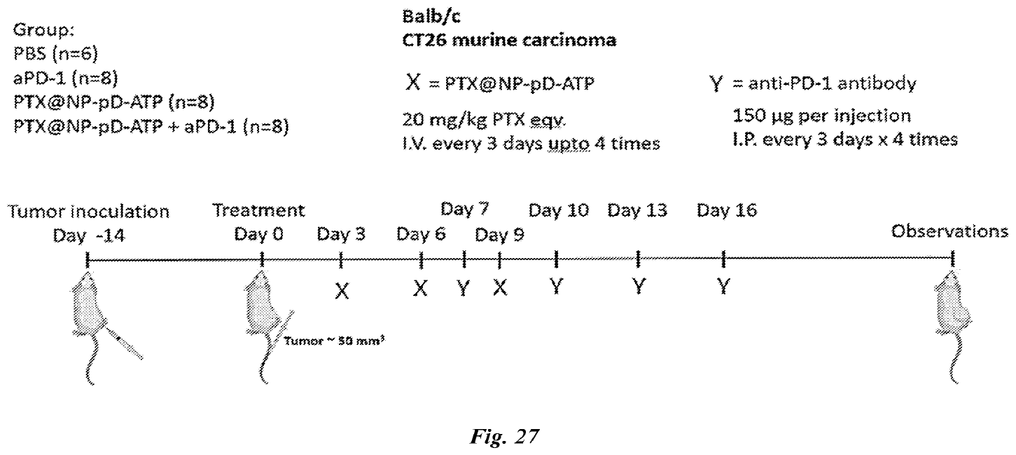

The systemic antitumor efficacy of PTX@NP-pD-ATP administered in combination with anti-PD-1 antibody was assessed with CT26 tumor bearing mice. Starting when the tumor reached a volume of 50 mm³, the mice were treated with a PTX equivalent of 20 mg/kg intravenously 4 times every 3 days. The mice additionally received sequential injections of anti-PD-1 antibody intraperitoneally 4 times every 3 days (FIG. 27).

More specifically, female BALB/c mice (4-6 weeks-old) were inoculated with 3×10⁵ CT26 cells subcutaneously in the upper flank of right hind limb. When the tumors grew to 50-100 mm³, the mice were randomly assigned to 4 groups and treated with PBS. PBS+anti-PD-1 antibodies. PTX@NP-pD-ATP, PTX@NP-pD-ATP+anti-PD-1 antibodies. PBS or PTX@NP-pD-ATP (PTX equivalent 20 mg/kg) were given intravenously every 3 days up to 4 times.

As the study was designed to downregulate immunosuppressive Treg cells in the tumor, the antibodies were injected 7 days after the initial injection of PTX, when T cells were activated following the antigen presentation. Indeed, providing a sequential injection has demonstrated a higher antitumor efficacy in other combination therapies. See. e.g., Messenheimer et al., Timing of PD-1 Blockade Is Critical to Effective Combination Immunotherapy with Anti-OX40, Clin Cancer Res 23: 6165-6177 (2017) and Kim et al., Sequential and Timely Combination of a Cancer Nanovaccine with Immune Checkpoint Blockade Effectively Inhibits Tumor Growth and Relapse. Angew Chem Int Ed Engl 59: 14628-14638 (2020). Accordingly, beginning 7 days after the initial injection of PBS or PTX@NP-pD-ATP, anti-PD-1 antibodies (150 μg per dose) were administered intraperitoneally every 3 days up to 4 times. Tumor growth was monitored.

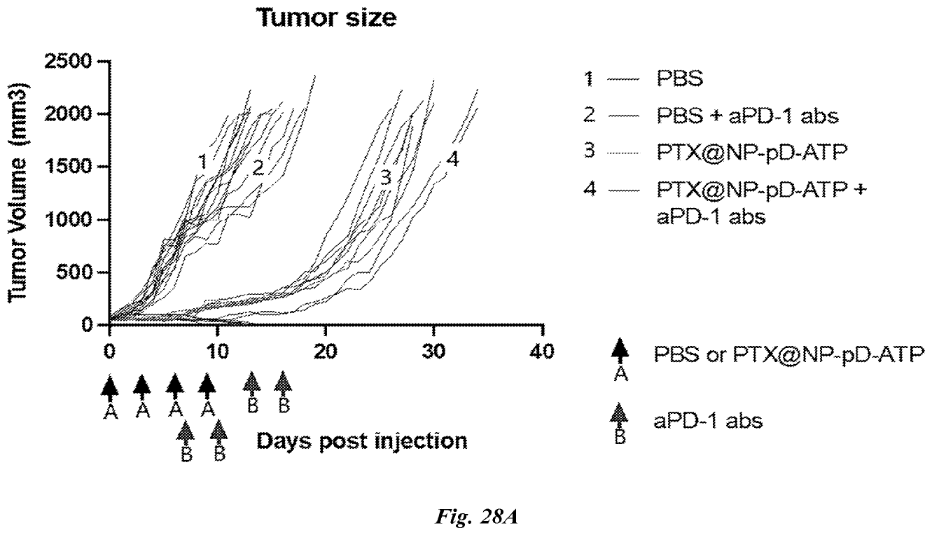

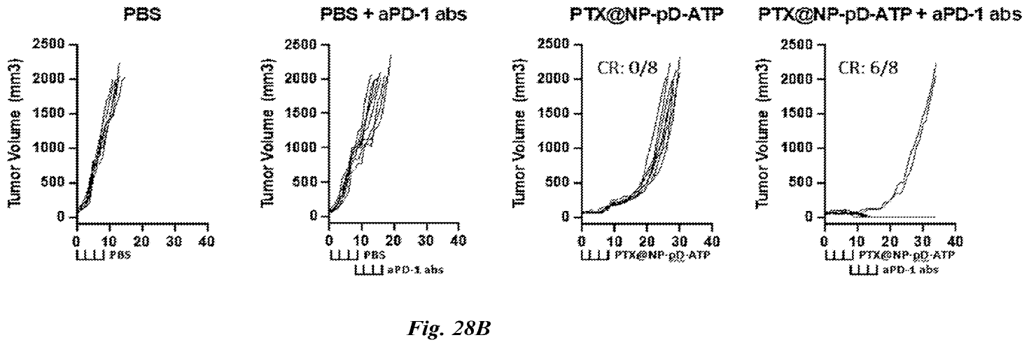

The combination of PTX@PLGA-pD-ATP and anti-PD-1 antibody significantly improved the outcome of tumor bearing mice, resulting in complete regression of tumor in 75% of the mice (6/8), and delayed tumor growth in the other two mice (FIGS. 28A and 28B).

As anti-PD-1 antibody was given 7 days after the PBS treatment when the tumors already have reached a volume of greater than 500 mm³, it is possible that anti-PD-1 antibody may not have worked because a large tumor can already exhibit a strong immunosuppressive TME. To observe how anti-PD-1 antibody acts against small size tumor (about 50-100 mm³ in volume), additional animals were inoculated with CT26 tumor as described above, and anti-PD-1 antibody was first given when the tumor volume was around 50-100 mm³ in volume. While anti-PD-1 antibodies suppressed the growth of the tumor compared to the control group (FIGS. 29A and 20B), they were not as effective as the combination therapy, with no complete regression of tumors.

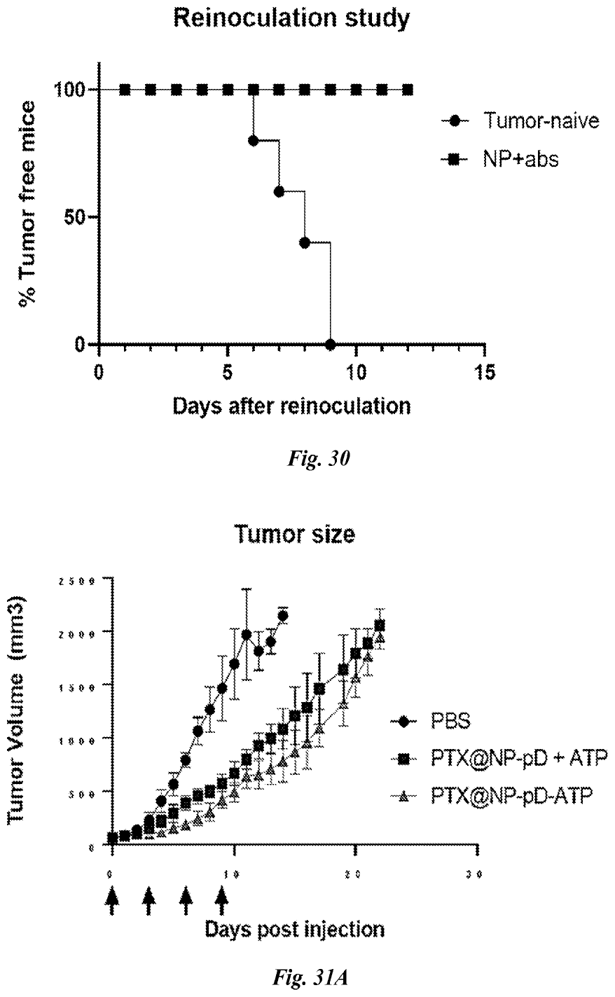

To investigate if the mice that survived the combination therapy had developed adaptive immunity against the CT26 tumor, the mice that reached complete remission were re-challenged with CT26 cells on the contralateral flank (per the previous protocol), and tumor growth was observed (FIG. 30). Tumors did not grow in survived mice: however, tumors did start to grow in age-matched naïve mice as soon as 7 days after the inoculation, supporting that tumor-free mice from the combination treatment group developed antitumor immunity against CT26.

### Example 19

### Antitumor Efficacy Against CT26 in Immunodeficient Mice

To confirm the improvement of the antitumor response was dependent on adaptive immunity, nude mice that lacked in mature T cell populations were also tested with PTX@NP-pD-ATP.

Primarily, female nude mice (4-6 weeks-old) were inoculated with 3×10⁵ CT26 cells subcutaneously in the upper flank of right hind limb. Once the tumor reached the volume of 50 mm³, the mice were randomly assigned to 3 groups and treated with an intravenous injection of 150 μL of PBS, PTX a NP-pD-ATP, or PTX@NP+ATP mixture every 3 days, 4 times total. The PTX dose was 20 mg/kg equivalent for all the PTX receiving group. Tumor growth was monitored.

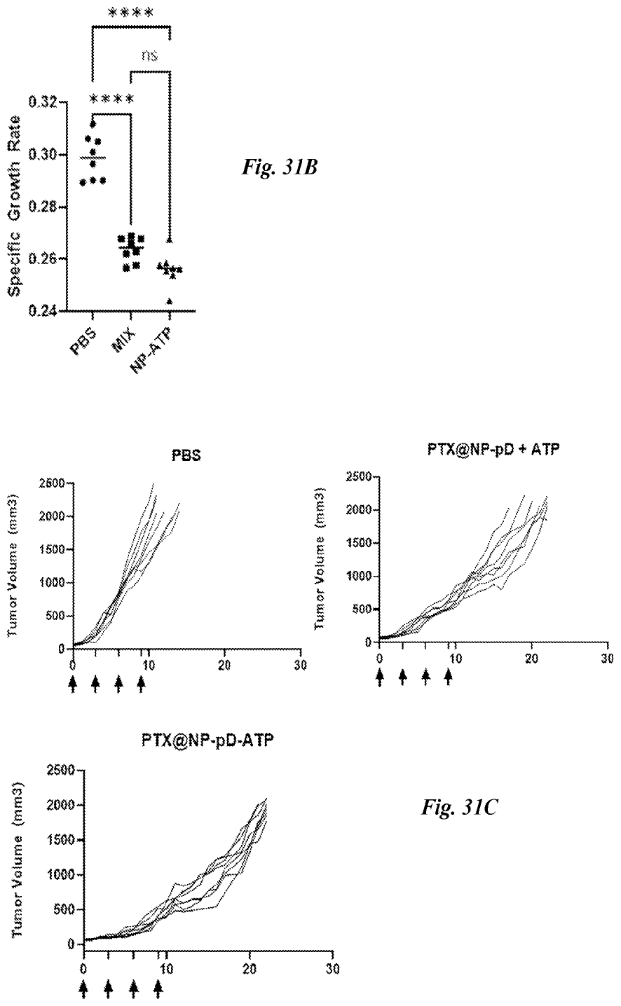

Unlike the immunocompetent model, PTX@NP-pD-ATP showed no difference in the tumor growth (specific tumor growth rate) from the mixture group (FIGS. 31A-31C), supporting that the activity of ATP coated nanoparticle depends on T cell activation.

### Example 20

ATP does not Affect the Accumulation of PTX in the Tumor

ATP has heretofore been utilized as a tumor targeting ligand to enhance the delivery of the cargo based on the overexpression of P2X7 receptors on cancer cells. See. e.g., Rajabnia and Meshkini, Fabrication of adenosine 5′-triphosphate-capped silver nanoparticles: Enhanced cytotoxicity efficacy and targeting effect against tumor cells, Process Biochemistry 65: 186-196 (2018). The test in nude mice (Example 19) suggests the activation of antitumor immunity by ATP decorated nanoparticles, but ATP conjugated to PLGA nanoparticles could also enhance the delivery of the PTX to CT26 or B16F10 tumors by targeting tumors (as compared to non-coated nanoparticle that are not targeted).

To screen if ATP decorated nanoparticles enhance the uptake of nanoparticle via P2X7 receptor interaction, Rhodamine B-labeled PLGA was utilized to form nanoparticles, and the surface of the Rhodamine B-labeled PLGA nanoparticles were non-coated (i.e. non-decorated), coated with polyethyleneimine (PEI), or coated with ATP. PEI was used as a positive control due to its known ability to promote cellular uptake non-specifically. The uptake of nanoparticles by tumor cells were analyzed via confocal microscopy and flow cytometry.

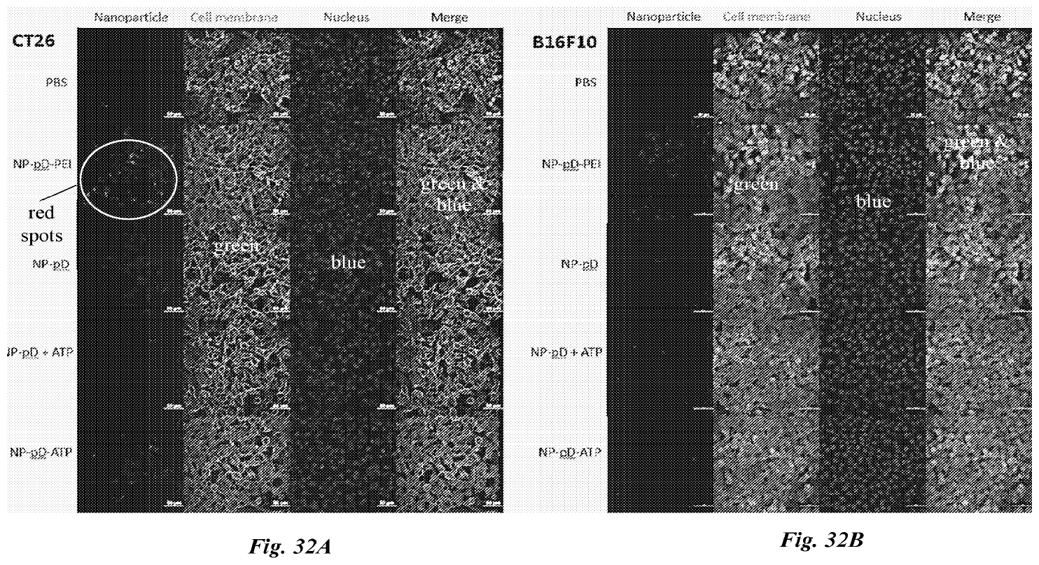

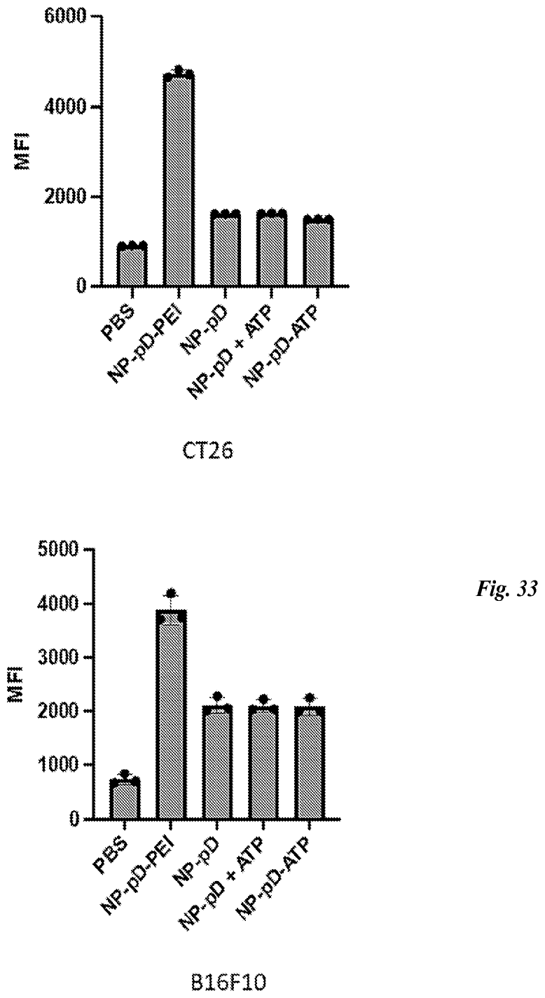

From the confocal microscopy, ATP-coated nanoparticles were not actively taken up by CT26 cells or B16F10 cells, whereas PEI-coated nanoparticles were taken up by tumor cells (FIGS. 32A and 32B). Flow cytometry results showed a consistent trend (FIG. 33). The in vitro results support ATP conjugation may not enhance the uptake of nanoparticles to the tumor.

To evaluate the targeting effect in vivo, CT26 tumor-bearing mice were treated with either a PTX@NP-pD+ATP mixture (i.e., the mixture or the mixture treatment group) or PTX@NP-pD-ATP, and the amount of PTX accumulation in the tumor was measured 24 hours after the first treatment. The 24-hour timepoint was chosen based on the literature findings that suggest the highest accumulation of nanoparticles in the tumor will occur 24 hours after the injection. See Hyun et al., Surface modification of polymer nanoparticles with native albumin for enhancing drug delivery to solid tumors, Biomaterials 180: 206-224 (2018) and Xu et al., Quinic Acid-Conjugated Nanoparticles Enhance Drug Delivery to Solid Tumors via Interactions with Endothelial Selectins, Small 14: e1803601 (2018).

Female BALB/c mice (4-6-week-old) were inoculated with 3×10⁵ CT26 cells subcutaneously in the upper flank of right hind limb. When the tumors grew to 100-150 mm³, the mice were randomly assigned to 3 groups and treated with PBS, PTX@NP-pD+ATP or PTX@NP-pD-ATP once (PTX equivalent 20 mg/kg). After 24 hours, tumors were harvested, and the amount of PTX in the tumor were measured after extraction.

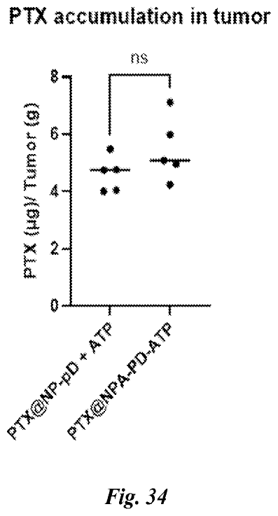

The amount of PTX in tumor was measured by HPLC with carbamazepine as an internal standard. The accumulation of PTX within the tumor was not different between mixture and PTX@NP-pD-ATP groups (FIG. 34). This result indicates that ATP-coated nanoparticles did not enhance the accumulation of PTX in CT26 tumor models. Therefore, the improved anti-tumor activity of PTX@NP-pD-ATP compared to PTX@NP-pD or PTX@NP-pD+ATP mixture was not due to the altered delivery of PTX to the tumor, but instead enhancement of antitumor immunity via surface-bound ATP, which recruits DCs and then activates T cells.

What is claimed is:

1. A nanoconstruct comprising:
a nanoparticle which has an exterior surface;
one or more therapeutic agents encapsulated within the nanoparticle; and
an immunoadjuvant modified polyphenol compound bound to the exterior surface of the nanoparticle.

2. The nanoconstruct of claim 1, wherein the polyphenol compound is selected from the group consisting of polymerized dopamine (pD), tannic acid, tannic acid-iron complex, gallic acid, ellagic acid, hydroxyhydroquinone, epigallocatechin, epicatechin gallate, epigallocatechin galate, and pyrogallol.

3. The nanoconstruct of claim 1, wherein the immunoadjuvant is selected from the group consisting of adenosine triphosphate, calreticulin, high motility group box 1, deoxyribonucleic acid, annexin A1, type I interferon, heat shock protein 70, and heat shock protein 90.

4. The nanoconstruct of claim 1, wherein the nanoparticle is selected from the group consisting of poly(lactic-co-glycolic acid) (PLGA), polycaprolactone, D-α-tocopherol polyethylene glycol 1000 succinate-PLGA conjugate, polylactic acid, PLGA-methoxy-polyethylene glycol, ethylene vinyl acetate, mesoporous silica, liposomes, nanocrystals, and polyphenol aggregates.

5. The nanoconstruct of claim 1, wherein the nanoparticle is PLGA.

6. The nanoconstruct of claim 1, wherein the one or more therapeutic agents is one or more chemotherapeutic agents.

7. The nanoconstruct of claim 1, wherein at least one of the one or more therapeutic agents is an immunogenic cell death inducer.

8. The nanoconstruct of claim 1, wherein the one or more therapeutic agents is oxaliplatin, carfilzomib, paclitaxel, mitoxantrone, bleomycin, doxorubicin, epirubicin, idarubicin, cyclophosphamide, or cardiac glycosides.

9. The nanoconstruct of claim 1, wherein the one or more therapeutic agents is carfilzomib or paclitaxel.

10. (canceled)

11. (canceled)

12. (canceled)

13. (canceled)

14. (canceled)

15. (canceled)

16. (canceled)

17. (canceled)

18. A method for treating a cancer in a subject, which method comprises administering to the subject:
a priming dose comprising a therapeutically effective amount of a nanoconstruct comprising:
a nanoparticle which has an exterior surface,
one or more therapeutic agents encapsulated within the nanoparticle, and
an immunoadjuvant modified polyphenol compound bound to the exterior surface of the nanoparticle; and
an immune checkpoint inhibitor, a tumor-targeting antibody, or a cancer vaccine, wherein the priming dose induces or enhances an anti-tumor immune response in the subject at a targeted site.

19. The method of claim 18, wherein the immunoadjuvant is selected from the group consisting of adenosine triphosphate, calreticulin, high motility group box 1, deoxyribonucleic acid, annexin A1, type I interferon, heat shock protein 70, and heat shock protein 90.

20. The method of claim 18, wherein the one or more therapeutic agents are selected from the group consisting of oxaliplatin, carfilzomib, paclitaxel, mitoxantrone, bleomycin, doxorubicin, epirubicin, idarubicin, cyclophosphamide, and cardiac glycosides.

21. The method of claim 18, wherein the nanoparticle is selected from the group consisting of poly(lactic-co-glycolic acid) (PLGA), polycaprolactone, D-α-tocopherol polyethylene glycol 1000 succinate-PLGA conjugate, polylactic acid, PLGA-methoxy-polyethylene glycol, ethylene vinyl acetate, mesoporous silica, liposomes, nanocrystals, and polyphenol aggregates.

22. The method of claim 18, wherein the nanoparticle is PLGA.

23. The method of claim 18, wherein the one or more therapeutic agents is/are chemotherapeutic agents.

24. The method of claim 18, wherein at least one of the therapeutic agents is an immunogenic cell death inducer.

25. The method of claim 18, wherein the polyphenol compound is selected from the group consisting of polymerized dopamine (pD), tannic acid, tannic acid-iron complex, gallic acid, ellagic acid, hydroxyhydroquinone, epigallocatechin, epicatechin gallate, epigallocatechin galate, and pyrogallol.

26. The method of claim 18, wherein the priming dose is administered at least four days prior to the immune checkpoint inhibitor, thereby treating cancer in the subject.

27. The method of claim 26, wherein the immune checkpoint inhibitor is an antibody or antibody fragment targeting PD-1 (e.g., nivolumab or pembrolizumab) or PD-L1 (e.g., atezolizumab, avelumab, or durvalumab), CTLA-4 (e.g., tremelimumab or ipilimumab), an anti-CD25 antibody (e.g., basiliximab), or decitabine (e.g., a demethylating agent to control T-cell exhaustion).

28. (canceled)

29. The method of claim 26, wherein the targeted site is a cancerous tissue or cell.

30. (canceled)

31. (canceled)

32. (canceled)

33. (canceled)

34. (canceled)

35. (canceled)

36. (canceled)
